SEQUENCE LISTING (4 sequences)

SEQ ID NO: 1 - DNA, 1221 bp, Escherichia coli
  source 1..1221: mol_type = other DNA
  misc_feature 1200..1221: function = His-tag
atgagccaaaactgcaacagcagcggtaacaagtacccgatcgtgctggttcacggtctgggtggcttcggccgtgatgaactgggtggcgtgatcaagtattggggtggcgtttacgacattcaagaatatctgaaaagccgtggttatgaggtgtacaccgttagcattggcccggtgagcagcaactgggatcgtgcgtgcgagctgtatgcgcagctggttggtggcaccgttgactatggtgcggcgcatgcggcgaagtatggtcacaaacgttatggccgtacctacccgggtctgatcccggatctgggcaagattgacccgaaaaccggtgaaatgaagaaagtgcacctgatcggtcacagcatgggtggccagaccattcgtaccctggttcaactgctggcggaaggctgcgaggaagagcgtaactacagccaggagaacgtgagcccgctgttcatgggtggcaacaactgggtgaagagcgttaccaccatcagcaccccgcacgatggcaccagcctggcggattactattacaacgacccgaaaagcgttacctggcgtcaacacctgatcggcctgattgcgagcaccccgctgagcgacaacagcaacctgaagtacgatctgaaactggaccagtggggtctgacctataagaaagaagagagcacctatgagtacctgaaacgtatcattaacagcagcttttggatgaccaaccgtgatctggcgaactacgacgtgagcatcgaaggcgcggcggagctgaacagctgggtgaaggcgcagccggatgtttattacttcagctgggcgacccaagcgaccaccaaaagccgttttagcgactaccaggtgccgatttggagcatgtatctggaaggctggcaaagcgcgtacttcatgggtagctatacccgtaacatcccgggcaagattccggttaccagcgagtggtggccgaacgatggtgcggtgaacgttatcagccagaacggcccgaaaattaacagcaacgatcgtatcattaactttgacggcaacccggtgccgggtgtttggaacttcatgggtatcatggacacctttgatcaccgtgacatcattggtctgggcacctactggaacccgtgcccgtggtatctgaagcacgttgaactgctggcgaaactgccgaccctcgagcaccaccaccaccaccactga

SEQ ID NO: 2 - DNA, 1221 bp, Escherichia coli
  source 1..1221: mol_type = other DNA
  misc_feature 1200..1221: function = His-tag
  variation 186: allele = C to T
  variation 662: allele = T to A
  variation 1130: allele = G to A
atgagccaaaactgcaacagcagcggtaacaagtacccgatcgtgctggttcacggtctgggtggcttcggccgtgatgaactgggtggcgtgatcaagtattggggtggcgtttacgacattcaagaatatctgaaaagccgtggttatgaggtgtacaccgttagcattggcccggtgagcagtaactgggatcgtgcgtgcgagctgtatgcgcagctggttggtggcaccgttgactatggtgcggcgcatgcggcgaagtatggtcacaaacgttatggccgtacctacccgggtctgatcccggatctgggcaagattgacccgaaaaccggtgaaatgaagaaagtgcacctgatcggtcacagcatgggtggccagaccattcgtaccctggttcaactgctggcggaaggctgcgaggaagagcgtaactacagccaggagaacgtgagcccgctgttcatgggtggcaacaactgggtgaagagcgttaccaccatcagcaccccgcacgatggcaccagcctggcggattactattacaacgacccgaaaagcgttacctggcgtcaacacctgatcggcctgattgcgagcaccccgctgagcgacaacagcaacctgaagtacgatctgaaactggaccagtggggtcagacctataagaaagaagagagcacctatgagtacctgaaacgtatcattaacagcagcttttggatgaccaaccgtgatctggcgaactacgacgtgagcatcgaaggcgcggcggagctgaacagctgggtgaaggcgcagccggatgtttattacttcagctgggcgacccaagcgaccaccaaaagccgttttagcgactaccaggtgccgatttggagcatgtatctggaaggctggcaaagcgcgtacttcatgggtagctatacccgtaacatcccgggcaagattccggttaccagcgagtggtggccgaacgatggtgcggtgaacgttatcagccagaacggcccgaaaattaacagcaacgatcgtatcattaactttgacggcaacccggtgccgggtgtttggaacttcatgggtatcatggacacctttgatcaccgtgacatcattggtctggacacctactggaacccgtgcccgtggtatctgaagcacgttgaactgctggcgaaactgccgaccctcgagcaccaccaccaccaccactga

SEQ ID NO: 3 - protein, 406 aa, Escherichia coli
  BINDING 401..406: note = His tag
MSQNCNSSGNKYPIVLVHGLGGFGRDELGGVIKYWGGVYDIQEYLKSRGYEVYTVSIGPVSSNWDRACELYAQLVGGTVDYGAAHAAKYGHKRYGRTYPGLIPDLGKIDPKTGEMKKVHLIGHSMGGQTIRTLVQLLAEGCEEERNYSQENVSPLFMGGNNWVKSVTTISTPHDGTSLADYYYNDPKSVTWRQHLIGLIASTPLSDNSNLKYDLKLDQWGLTYKKEESTYEYLKRIINSSFWMTNRDLANYDVSIEGAAELNSWVKAQPDVYYFSWATQATTKSRFSDYQVPIWSMYLEGWQSAYFMGSYTRNIPGKIPVTSEWWPNDGAVNVISQNGPKINSNDRIINFDGNPVPGVWNFMGIMDTFDHRDIIGLGTYWNPCPWYLKHVELLAKLPTLEHHHHHH

SEQ ID NO: 4 - protein, 406 aa, Escherichia coli
  BINDING 401..406: note = His tag
  VARIANT 221: note = Leu to Gln (L to Q)
  VARIANT 337: note = Gly to Asp (G to D)
MSQNCNSSGNKYPIVLVHGLGGFGRDELGGVIKYWGGVYDIQEYLKSRGYEVYTVSIGPVSSNWDRACELYAQLVGGTVDYGAAHAAKYGHKRYGRTYPGLIPDLGKIDPKTGEMKKVHLIGHSMGGQTIRTLVQLLAEGCEEERNYSQENVSPLFMGGNNWVKSVTTISTPHDGTSLADYYYNDPKSVTWRQHLIGLIASTPLSDNSNLKYDLKLDQWGQTYKKEESTYEYLKRIINSSFWMTNRDLANYDVSIEGAAELNSWVKAQPDVYYFSWATQATTKSRFSDYQVPIWSMYLEGWQSAYFMGSYTRNIPGKIPVTSEWWPNDGAVNVISQNGPKINSNDRIINFDGNPVPGVWNFMGIMDTFDHRDIIGLDTYWNPCPWYLKHVELLAKLPTLEHHHHHH

(19) United States
(12) Patent Application Publication
(10) Pub. No.: US 2026/0265713 A1
(43) Pub. Date: Sep. 10, 2026

(54) THERMOSTABLE ENZYMES

(71) Applicant: ENZIDE TECHNOLOGIES LTD, West Perth, Western Australia (AU)

(72) Inventors: Colin SCOTT, West Perth, Western Australia (AU); Hafna AHMED, West Perth, Western Australia (AU)

(21) Appl. No.: 19/125,214

(22) PCT Filed: Oct. 25, 2023
(86) PCT No.: PCT/AU2023/051063

§ 371 Date: Apr. 28, 2025

(30) Foreign Application Priority Data
Oct. 28, 2022 (AU) 2022903216

(51) Int. Cl. C12N 9/20

(52) U.S. Cl. CPC C12N 9/20; C12Y 301/01003

(57) ABSTRACT

Novel enzyme variants of the naturally occurring wild-type Thermosyntropha lipolytica Tl_Est47 are provided with improved thermostable properties which do not negatively affect the stability or activity of the enzyme variants at a range of temperatures or pH values.

### REFERENCE TO ELECTRONIC SEQUENCE LISTING

The application contains a Sequence Listing which has been submitted electronically in.XML format. The.XML file, created on Jun. 10, 2025, is named “990724.1160.xml” and is 11,081 bytes in size. The Sequence Listing, contained within this.XML file, is part of the specification and is hereby incorporated by reference herein in its entirety.

### FIELD OF THE INVENTION

The invention relates to novel variants of the Thermosyntropha lipolytica Tl_Est47 enzyme with improved thermostable properties when compared to the naturally occurring wild-type Thermosyntropha lipolytica Tl_Est47.

### BACKGROUND

The following discussion of the background art is intended to facilitate an understanding of the present invention only. It should be appreciated that the discussion is not an acknowledgement or admission that any of the material referred to was part of the common general knowledge as at the priority date of the application.

Enzymes have been used for many years in products in the detergent, textile, and starch industries, as well as in food production including for cheese, bread, beer, wine, leather and linen amongst others. These industrial processes utilize enzymes produced by and isolated from specific microorganisms, or by enzymes present in natural products (e.g. papaya fruit).

With more recent advancements in protein engineering, mutant enzymes for established applications or new custom made enzymes for areas of applications where enzymes have not been previously used have been developed. Of those enzymes used in industrial processes, more than half are from fungi, over a third from bacteria, and the remainder from animal and plant sources. Recombinant DNA techniques enable the isolation and cloning of genes encoding for enzymes from all possible sources and high yield heterologous expression. The result has seen increased production levels and enzymes from bacterial strains not originally suited for industry including Aspergillus, Saccharomyces, and Bacillus, amongst others.

The global market for industrial enzymes is estimated to be currently around USD10 billion and some of the more commonly utilised enzymes include lipases, polyphenol oxidases, lignin peroxidase, horseradish peroxidase, amylase, nitrite reductase, and urease.

Enzymes are selected for use in these industrial processes for the reactions they cause and catalyse, as well as their various characteristics in working within the environment of the industrial processes, for example, properties that enable the enzyme to maintain enzymatic activity at various required temperature and pH environments.

There are substantial benefits with enzymes having high levels of thermostability for use in many of these industrial processes which involve high temperatures at specific stages, or in products for use in conditions that include high temperatures.

Thus, there is a growing need for enzymes with thermostable characteristics at high temperatures for use in products and specific industrially useful applications and processes.

### SUMMARY OF INVENTION

The inventors have engineered novel polypeptides which are variants of Thermosyntropha lipolytica Tl_Est47 with improved thermostability in terms of maintaining enzymatic activity at higher temperatures, when compared to naturally occurring (wild-type) Thermosyntropha lipolytica Tl_Est47.

In a first aspect, the invention provides novel polypeptides comprising:
i) an amino acid sequence as provided in SEQ ID NO:4,
ii) an amino acid sequence which is at least 90% identical to i), or
iii) a biologically active fragment of ii).

In an embodiment, the novel polypeptides of the invention comprise one or both of:
i) a glutamine (Q) at a position corresponding to amino acid number 221 of SEQ ID NO: 4; and
ii) an aspartic acid (D) at a position corresponding to amino acid number 377 of SEQ ID NO: 4.

In an embodiment, the novel polypeptides of the invention comprises a fusion protein comprising at least one other polypeptide sequence.

In an embodiment, the invention provides an isolated and/or exogenous polynucleotide of the invention comprising a sequence selected from:
a. a sequence of nucleotides as provided in SEQ ID NO: 2;
b. a sequence of nucleotides encoding novel polypeptides of the invention; or
c. a sequence of nucleotides complementary to either i) or ii).

In an embodiment, the invention provides an isolated and/or exogenous polynucleotide of the invention as herein described, wherein the polynucleotide is operably linked to a promoter capable of directing expression of novel polypeptides of the invention in a cell.

In an embodiment, the invention provides an isolated and/or exogenous polynucleotide of the invention as herein described, wherein the polynucleotide is operably linked to a promoter capable of directing expression of novel polypeptides of the invention in an expression host cell.

In an embodiment, the invention provides a vector comprising a polynucleotide of the invention as herein described.

In an embodiment, the invention provides a nucleic acid construct or expression vector comprising a polynucleotide of the invention as herein described.

In an embodiment, the invention provides a nucleic acid construct or expression vector comprising the polynucleotide of the invention as herein described, wherein the polynucleotide is operably linked to one or more control sequences that direct the production of novel polypeptides of the invention in an expression host cell.

In an embodiment, the invention provides a recombinant expression host cell comprising a polynucleotide encoding novel polypeptides of the invention, wherein the polynucleotide is operably linked to one or more control sequences that direct the production of the polypeptide.

In an embodiment, the invention provides a host cell comprising a polynucleotide of the invention as herein described.

In an embodiment, the host cell preferably comprises a bacterial cell, a fungal cell, or a plant cell.

In an embodiment, the invention provides a transgenic non-human organism comprising at least one herein described host cell.

In an embodiment, the invention provides an extract of a herein described host cell, wherein the extract comprises a polypeptide comprising:
a. an amino acid sequence as provided in SEQ ID NO:4;
b. an amino acid sequence which is at least 90% identical to i), or
c. a biologically active fragment of ii).

In an embodiment, the invention provides an extract of a herein described host cell, wherein the polypeptide comprises one or both of:
a. a glutamine (Q) at a position corresponding to amino acid number 221 of SEQ ID NO:4; and
b. an aspartic acid (D) at a position corresponding to amino acid number 377 of SEQ ID NO:4.

In an embodiment, the invention provides a composition comprising novel polypeptides of the invention, and one or more acceptable carriers.

In an embodiment, the invention provides a composition comprising a herein described extract, and one or more acceptable carriers.

In an embodiment, the invention provides a method of producing novel polypeptides comprising:
a. an amino acid sequence as provided in SEQ ID NO:4;
b. an amino acid sequence which is at least 90% identical to i), or
c. a biologically active fragment of ii).

In an embodiment, the novel polypeptides preferably comprise one or both of:
a. a glutamine (Q) at a position corresponding to amino acid number 221 of SEQ ID NO:4; and
b. an aspartic acid (D) at a position corresponding to amino acid number 377 of SEQ ID NO:4.

In an embodiment, the invention provides a method of producing novel polypeptides of the invention, comprising cultivating a recombinant expression host cell comprising novel polypeptides of the invention, wherein the polynucleotide is operably linked to one or more control sequences that direct the production of the novel polypeptides of the invention under conditions conducive for production of the polypeptide.

In an embodiment, the method preferably comprises recovering the novel polypeptides of the invention.

In an embodiment, the invention provides an enzyme comprising a novel polypeptide of the invention.

In an embodiment, the invention provides a thermostable enzyme comprising a novel polypeptide of the invention.

In an embodiment, the invention provides a hyperthermophilic enzyme comprising a novel polypeptide of the invention.

In an embodiment, the invention provides a herein described enzyme, wherein the enzyme maintains enzymatic activity at a temperature higher than the temperature at which an enzyme comprising a wild-type Thermosyntropha lipolytica Tl_Est47 polypeptide comprising an amino acid sequence as provided in SEQ ID NO:3 loses substantially all enzymatic activity.

In an embodiment, the herein described enzyme maintains enzymatic activity at 90° C. or higher.

In an embodiment, the herein described enzyme maintains some enzymatic activity at 95° C. or higher.

In an embodiment, the herein described enzyme comprises esterase activity.

In an embodiment, the invention provides a herein described enzyme comprising lipase activity.

In an embodiment, the invention provides a novel polypeptide of the invention which comprises a polypeptide variant of wild-type Thermosyntropha lipolytica Tl_Est47, wherein the polypeptide variant of wild-type Thermosyntropha lipolytica Tl_Est47 comprises an amino acid sequence as provided in SEQ ID NO:3.

In an embodiment, the invention provides a polynucleotide of the invention as herein described which is capable of expressing a polypeptide variant of wild-type Thermosyntropha lipolytica Tl_Est47, wherein the wild-type Thermosyntropha lipolytica Tl_Est47 comprises an amino acid sequence expressed by an isolated and/or exogenous polynucleotide comprising a sequence selected from a sequence of nucleotides as provided in SEQ ID NO: 1.

### BRIEF DESCRIPTION OF FIGURES

The present invention will now be described, by way of example, with reference to the accompanying drawings, in which:

FIG. 1. Illustration showing unrooted maximum-likelihood phylogenetic tree of the bacterial lipase family 1.5 generated using IQ-tree and visualised by iTOL v5. UF-Boot values are shown for nodes with SH-aLRT support values >=80%. Report of chemical analysis of macroalgae hydrolysate.

FIG. 2. Esterase activity of the lipase homologues with pNP substrates. (A) Bar graph of an activity test using 5 μl of cell culture with 0.75 mM pNP acetate to confirm protein expression. Error bars represent SEM from 3 repeat experiments. (B) Photograph of an SDS-PAGE gel analysing the purity of the proteins obtained from small-scale nickel affinity purification. The expected size of the bands are indicated at the bottom of the gel. (C). Bar graph showing a comparison of the activity of 10 nM purified protein with pNP substrates of different lengths. Error bars indicate SEM from 2 repeat experiments.

FIG. 3. Line graph showing residual activity of the thermostable proteins with pNP acetate and pNP propionate. Samples were heated at the designated temperature for 10 min and cooled at 4° C. before measuring the residual activity, which is provided as a percentage of activity of an unheated sample. The 6 enzymes shown were identified from an initial assay with all 10 enzymes presented in this work. Error bars represent SEM from 3 repeat experiments.

FIG. 4. Graph showing EqAD activity on 185 μl of supernatant from a reaction containing 100 nM Cl_EstA and 5 mg/mL PBAT that was incubated at 40° C. for 48 hr. The EqAD reaction contained 250 μM NAD+ and 0.1 U/mL of EqAD in a final reaction volume of 200 μL, and the reaction progress was measured by following the change in absorbance at 340 nm upon NADH++H⁺production.

FIG. 5. Thermostability of the P4G12 Tl_Est47 mutant measured using protein expressing cell culture. (A) Individual graphs from plates screened for activity by heating overnight cultures heated for ten minutes at 89° C., allowed to cool then tested for activity against paranitrophenyl butyrate. (B) Line graph showing residual activity of the improved variant TL_Est47 compared with wildtype after heating at temperatures between 75° C. and 99° C. for 10 minutes, allowed to cool then tested for activity against paranitrophenyl butyrate.

FIG. 6. Characterisation of WT Tl_Est47 and its L221Q and G377D mutant that demonstrated increased thermostability. (A) Line graph showing esterase activity of WT and mutant proteins measured using pNP butyrate as a substrate. (B) Table showing kinetic parameters for WT and mutant proteins for curves shown in (A).

FIG. 7. Line graph showing thermostability of purified WT and mutant Tl_Est47 in LB media. Reactions to measure residual activity contained 200 nM enzyme and 300 μM pNP butyrate, and the amount of pNP liberated was measured at an absorbance of 405 nm.

FIG. 8. Table showing Log of conditions in the 2 litre fermenter.

FIG. 9. Table showing Log of conditions in the 500 mL flask.

FIG. 10. Graph showing alcohol dehydrogenase activity from PBSA incubated with wild-type Tl_Est47 between pH 7 and pH 9 (Tris buffer).

FIG. 11. Graph showing alcohol dehydrogenase activity from PBSA incubated with Tl_Est47 variant between pH 7 and pH 9 (Tris buffer).

FIG. 12. Graph showing alcohol dehydrogenase activity from PBSA incubated with wild-type Tl_Est47 at between 4 and 37° C.

FIG. 13. Graph showing alcohol dehydrogenase activity from PBSA incubated with the Tl_Est47 variant at between 4 and 37° C.

FIG. 14. SDS PAGE gel of whole cell and soluble after 2 h, 4 h, 5.5 h and 22 h incubation time post-induction either in flask or in fermenters (blue arrow indicates Tl_Est47).

FIG. 15. The FPLC trace and SDS-PAGE gels of (A) the pellet from the 500 ml Flask culture and (B) the 2L fermentation. The absorbance at 280 nM is shown by the blue line. The SDS/PAGE shows the molecular size marker (M, sizes given to left of gels), whole cell (W) and soluble(S) fractions. 1-9 corresponds to the fractions from the FPLC trace analysed by SDS/PAGE.

### DESCRIPTION OF PREFERRED EMBODIMENTS

In order to provide a more precise understanding of the person matter of the invention, features of the invention will now be discussed with reference to the following preferred embodiment or embodiments.

Sequence retrieval and sequence similarity network (SSN) generation.

In an initial search of potential enzymes with improved thermostability, the sequences of CI_EstA (Accession no: WP_011948553.1), CI_EstB (Accession no: WP_011986581.1) and PfL1 (Accession no: EIW29778.1) were used to perform a pBLAST search against the NCBI Refseq_Select_proteins database using default parameters. All sequence identified with e-values >0.005 were retrieved and submitted to the Enzyme Function Initiative Enzyme Similarity Tool (EFI-EST) to generate a sequence similarity network (SSN) that only contained sequences of lengths between 200-1000 amino acids, with an initial alignment score cut-off of 7. In an SSN, nodes represent individual proteins and edges represent the alignment score, which is calculated by the EFI-EST algorithm using the bit-score from performing an all-vs-all BLAST of the provided protein sequences. The alignment score is close in magnitude to the negative logarithm of the BLAST E-value that approximates protein similarity.

The SSN was visualised using Cytoscape 3.9.0 by applying a yfile Organic Layout. The clustering was analysed by gradually increasing the alignment score cut-off followed by re-application of the layout, until no major changes were observed with a large jump in the cut-off value, which was from alignment scores of 20 through to 40 in this SSN. This visual cluster analysis method allows the identification of potentially iso-functional groups from other related proteins. Only sequences belonging to the large cluster that contained the query proteins were then used for further SSN and phylogenetic analysis. Further alignment score cut-off values and subsequent yfiles organic layout re-application was performed to visualise clades within the larger protein family.

Phylogenetic Analysis to Identify Sequences from Thermophiles and Extremophiles

The refined sequence set from the SSN was then aligned using the structure-based protein sequence alignment algorithm PROMALS3D with the structures of CI_EstA (PDB ID: 5AH1) and PfL1 (PDB ID: 5AH0) provided as templates. Poorly aligned regions at the N- and C-terminus as well as long insertions in otherwise well aligned sequences were deleted, and sequences with poor overall alignment or large deletions were removed entirely. This curated sequence set was re-aligned using MUSCLE using the EMBL-EBI webserver (https://www.ebi.ac.uk/Tools/msa/muscle/) with default settings. The resulting alignment was further trimmed to remove poorly aligned regions at the N-terminus as well as any large gaps or insertions, before being used to generate an initial approximate maximum-likelihood (ML) tree using FastTree2.1 with the WAG+CAT evolutionary model. The multiple sequence alignment (MSA) was further curated to remove any long branches along with highly similar sequences with very short branch lengths, and the process was repeated until the SH-like local support values were >80% for most nodes. The resulting alignment was used to infer a maximum likelihood tree using the IQ-TREE webservice (https://www.hiv.lanl.gov/content/sequence/IQTREE/iqtree.html). The calculated best evolutionary model used was as follows: LG model+FreeRate heterogeneity (#rate categories=8)+ML optimized AA frequencies from the data. Ultrafast bootstrap (UF-Boot) values and SH-aLRT branch test values were calculated for 1000 replicates. The tree was visualised and annotated using the Interactive Tree Of Life (iTOL) webtool (https://itol.embl.de/).

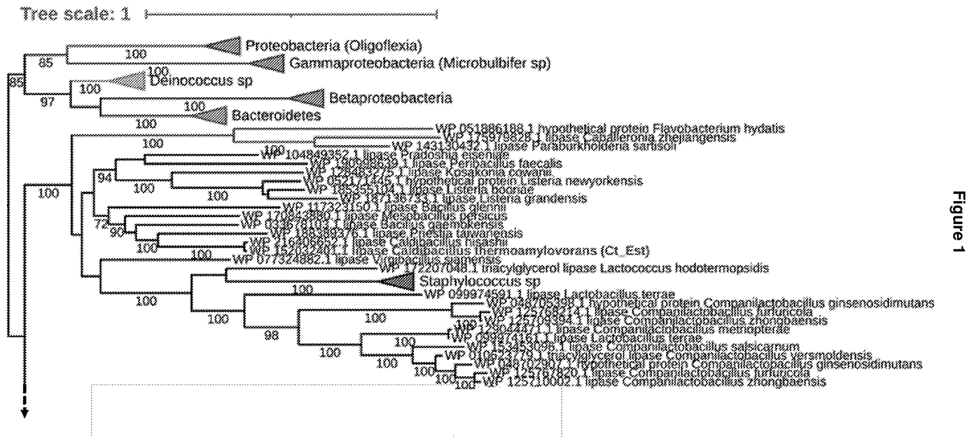

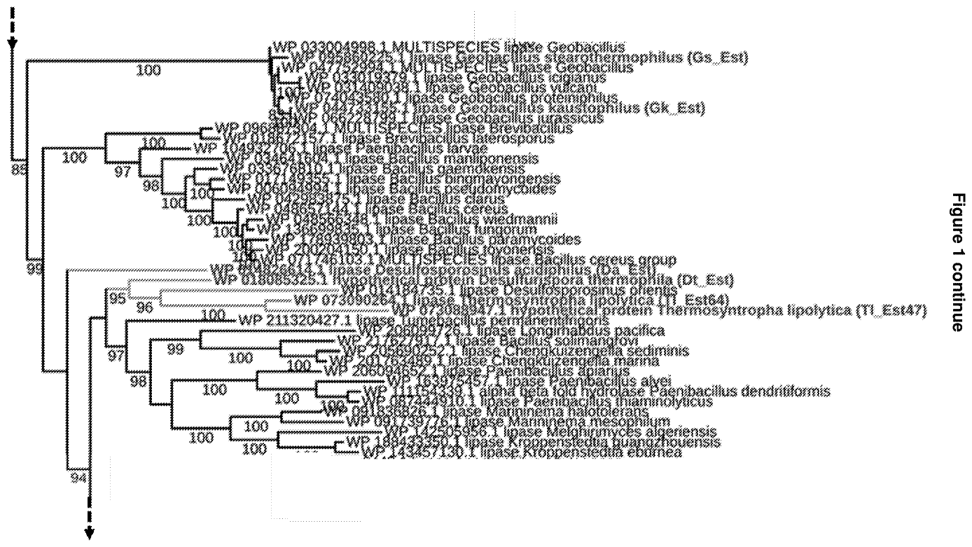

Sequences from bacterial lipase family 1.5 curated using the SSN were used to generate a maximum-likelihood phylogenetic tree (FIG. 1). This analysis supports observations made from the SSN, where CI_EstA, CI_EstB and PfL1 belonged to a large clade containing proteins from bacteria of the Clostridiaceae family. Within this clade, two sub-clades contain each CI_EstA and Cl_EstB, suggesting that they arose from a gene duplication event in the last common ancestor of the Clostridiaceae family. Like in the SSN, horizontal gene transfer from the CI_EstA clade to the last common ancestor of Pelosinus sp is supported in the phylogenetic tree, resulting in PfL1. The sequences from Clostridiaceae are the most closely related to those from Bacilli of the families Thermoactinomycetaceae, Paenibacillaceae, and Alicyclobacillaceae as also observed on the SSN. Among these Clostridia and Bacilli are also sequences from thermophilic and acidophilic Clostridia of the families Peptococcaceae and Syntrophomonadaceae. Distinct clades were observed for Bacillus sp and Geobacillus sp as well as a third large clade containing Bacilli of the orders Bacillales (such as Bacillus sp and Caldibacillus sp) and Lactobacillies. The sequences from all other phyla except for Firmicutes form a distinctly separate clade, except for 3 sequences from Betaproteobacteria and Bacteroidetes that a likely to have been acquired through horizontal gene transfer.

Based on the phylogenetic tree and SSN, further functional analysis was limited to sequences from Clostridia and Bacilli of the order Bacillales, as they demonstrated the closest homology to CI_EstA, CI_EstB and PfL1. Sequences were selected from 6 thermophiles and 1 acidophile namely: Ct_Est from Caldibacillus thermoamylovorans (accession: WP_152032401.1), Gk_Est from Geobacillus kaustophilus (accession: WP_044733155.1), Gs_Est from Geobacillus stearothermophilus (accession: WP_095860225.1), Tl_Est47 from Thermosyntropha lipolytica (accession: WP_073088947.1), Tl_Est64 from Thermosyntropha lipolytica (accession: WP_014826614.1), Dt_Est from Desulfurispora thermophila (accession: WP_018085325.1) and Da_Est from Desulfosporosinus acidophilus (accession: WP_014826614.1).

The Polyesterase Homologues Express in E. coli and Demonstrate Activity with pNP Substrates.

### Protein Expression

The 7 identified protein sequences of interest along with the sequences of CI_EstA, Cl_EstB and PfL1 were input into the SignalP-5.0 webserver, and the signal sequence predicted was trimmed before designing the expression vectors that were ordered from GenScript (Singapore). Each truncated sequence was placed between the Nde1 and Xho1 restriction sites in pET-29b (+) such that the expressed protein contains an N-terminal His-tag. The plasmids were transformed into NEB T7 express cells (New England Biolabs) with the manufacture recommended protocol and plated on Luria Broth (LB) agar plates containing 50 μg/mL of kanamycin. The plates were incubated over night at 37° C. and stored at 4° C. for a maximum of 2 weeks. A negative control plasmid (gfasPurple-S125R-F162R-V44A-L123T_pETcc2) was also transformed and plated on an LB agar plate containing 100 μg/mL of ampicillin.

For protein expression, single colonies from each strain were inoculated into 10 ml of auto-induction media (5 g yeast extract, 20 g tryptone, 85.5 mM NaCl, 22 mM KH₂PO₄, 42 mM Na₂HPO₄, 0.6% glycerol, 0.05% glucose and 0.2% lactose) containing 50 μg/ml of kanamycin (100 μg/mL of ampicillin for the control strain) in 50 ml tubes. Cultures were grown for 3-6 hours at 37° C. while shaking at 200 rpm, followed by overnight incubation at 30° C. The resulting cultures were either stored at 4° C. for up to 2 weeks for whole cell assays or spin down at 4000× g for 10 mins at 4° C., where the supernatant was discarded, and the resulting pellet was stored at −20° C. until protein purification.

The selected proteins were expressed with an N-terminal His-tag in E. coli strain NEB T7 Express. Protein profiles of the whole cell fraction (soluble and insoluble protein) and soluble fraction isolated from the cell cultures were evaluated on an SDS-PAGE gel.

Testing for Protein Expression with SDS-PAGE

500 μL from each culture was spun down at 4000× g at 4° C. for 10 mins in 1.5 mL microfuge tubes and the supernatant was discarded. The resulting cell pellet was resuspended in 100 μL of lysis solution (50 mM Tris pH 8, 1×BugBuster Protein Extraction Reagent (Millipore) and ~33 nL DNAse I), and left on ice for about 10 mins. After lysis, 5 μL from each sample was mixed with 10 μL of 50 mM Tris H 8 and 5 μL of 4×NuPAGE™ LDS Sample Buffer (Invitrogen). The rest of the samples were spun down at 20,000×g for 10 mins at 4° C., and 15 μL of the supernatant was mixed with 5 μL of 4×NuPAGE™ LDS Sample Buffer (Invitrogen). The samples were heated at 90° C. for 3 mins and loaded onto pre-cast NuPAGE™ 4-12% Bis-Tris gels (Invitrogen) and run for 30-40 min at 150 V in MES SDS running buffer (Invitrogen). Gels were stained with AcquaStain Protein Gel Stain (Bulldog) for 30 mins and de-stained in water.

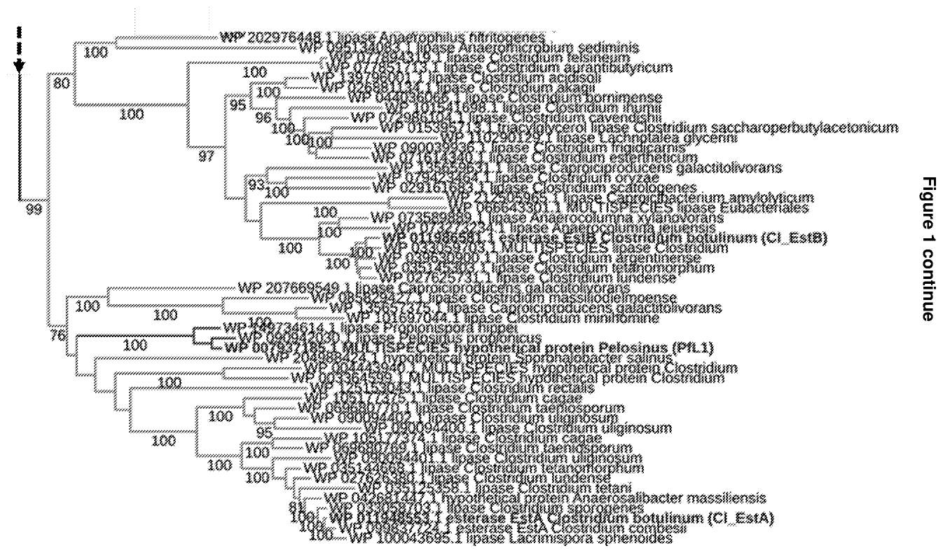

Clear bands were only observed for CI_EstA, CI_EstB, Dt_Est and Tl_Est64 due to a strong background band at the expected size that is also observed in the negative control. Hence to confirm protein expression, an initial esterase activity assay was performed with p-Nitrophenyl (pNP) acetate (FIG. 2A). Activity was detected for all 10 proteins, confirming their successful expression in E. coli.

To compare the relative activities of the proteins, the proteins were partially purified by small-scale nickel affinity chromatography (FIG. 2B).

### Protein Purification

For small scale purification, the pellets from 10 mL cultures were resuspended each in 1 ml of lysis buffer containing 50 mM Tris, 300 mM NaCl at pH 8 and transferred to 2 ml microfuge tubes. Cells were lysed by sonication (Fisher Scientific, 5 s pulse, 1 s break, 30 s, 3 times). The lysates were then spun at 20,000×g for 20 min and 4° C. and the supernatant was loaded onto NEBExpress® Ni Spin Columns (New England biolabs) that were pre-washed with 250 μL of the same lysis buffer. The columns were washed with 750 μL total of wash buffer (50 mM Tris, 300 mM NaCl, 5 mM imidazole, pH 8) and the samples were eluted with 2×200 μL of elution buffer (50 mM Tris, 300 mM NaCl, 500 mM imidazole, pH 8). From each eluent, 15 μL was mixed with 5 μL of 4×NuPAGE™ LDS Sample Buffer (Invitrogen) for SDS_PAGE analysis as described above. Purified protein were stored at 4° C. for up to 3 weeks.

Bands of approximately 90% purity or greater were observed for CI_EstA, PfL1, Da_Est, Dt_Est, Tl_Est47 and Tl_Est64. Although only ~40-50% purity was achieved for Cl_EstB and Ct_Est, clear bands for the protein of the expected sizes were still observed. In contrast, Gs_Est and Gk_Est had very low level, barely detectable protein purified using this protocol, even though pNP acetate activity was observed from the whole cell samples.

Esterase Activity Assay with pNP-Substrates.

The purified protein was then used to characterise the substrate preference of these proteins, comparing activities with pNP acetate, pNP propionate, pNP butyrate, pNP velarate and pNP octonoate (FIG. 2C).

To check activity of the proteins with pNP substrates, 90 μL of 50 mM Tris (pH 8.0) was first mixed with either 5 μL of cell culture for whole cell assays, or 5 μL of 200 nM purified protein. To start the reaction, 5 μL of 15 mM pNP-acetate, pNP-propionate, pNP-butyrate, pNP-velerate or pNP-octonoate in 100% methanol was added, such that the final reaction contained 5% methanol. The absorbance at 405 nm were measured over 10 min at 3-4 s intervals and the rate at the linear portion of the curve (usually within the first 30-100 s) was used to calculate the amount of pNP produced, with an extinction coefficient of 16,853 M-1 cm 1 with a pathlength of 0.25 cm.

A general trend of increased activity with increasing substrate length was observed for all proteins, except CI_EstA, which showed the opposite trend with the best activity with pNP acetate. Dt_Est showed the highest activity with the longest substrate tested. Tl_Est64 showed the highest activity from all the proteins with pNP butyrate and pNP velarate. As expected, CI_EstB, Ct_Est, Gk_Est and Gk_Est that purified poorly showed very low activity with all substrates. Da_Est from an acidophilic organism only showed slight activity with pNP octonoate and no activity with any other substrate, despite the purification being successful (FIG. 2B). It also showed only slight activity with pNP acetate when the cell culture was tested.

### CI_EstA, PfL1, Dt_Est, Tl_Est47 and Tl_Est64 are Highly Thermostable.

Next, the thermostability of these enzymes was tested by testing for the residual activity of cell cultures that were heated at various temperatures for 10 min.

For each enzyme (and the negative control culture), 8×15 μL of cell culture was added to 0.2 ml tubes in a strip and heated to various temperatures for 10 min using the temperature gradient function on a thermocycler (instrument). The tubes were then cooled on ice for another 10 mins. Residual activity was measured using the pNP-substrate assay described above with either pNP-acetate, pNP-propionte or pNP-butyrate. 5 μL of the heated and cooled cell culture samples were used, and a positive control containing 5 μL of unheated cell culture and a negative control containing 5 μL of buffer was also set up for each protein. Reaction rates in mOD/min were obtained and used to calculate the percentage residual activity as compared to the unheated positive control sample.

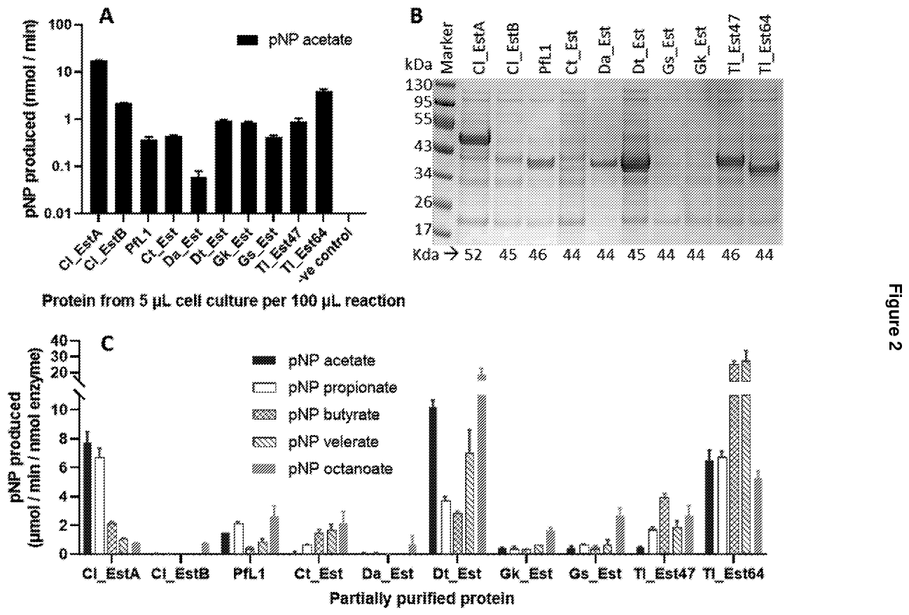

An initial test demonstrated clear residual activity with pNP acetate for CI_EstA, PfL1, Dt_Est, Gk_Est, Tl_Est47 and Tl_Est64 after heating at more than 60° C. Cells expressing these proteins were further evaluated over a range of temperatures to estimate their melting temperatures (Tm) (FIG. 3). The highest temperatures where at least 10% of residual activity was detected with pNP acetate were as follow: >10% for Dt_Est, Pfl1, and Tl_Est64 after heating at 78° C., >50% for CI_EstA after heating at 70° C., and >20% for Gk_Est after heating at 65.75° C.

Interestingly, Tl_Est47 showed >10% residual activity with pNP acetate even after heating at 85° C., and this was evaluated further by repeating the assay for this enzyme using pNP propionate. While Tl_Est47 has barely detectable activity with pNP acetate under these reaction conditions, the rate of reaction is increased by ~4-fold with pNP propionate as a substrate (FIG. 3), increasing the assay sensitivity. For Tl_Est47, we observed >10% residual activity with pNP propionate after heating at 90° C., making it the most thermostable enzyme evaluated in these experiments.

### Evolution of Tl_Est47B for Improved Thermostability.

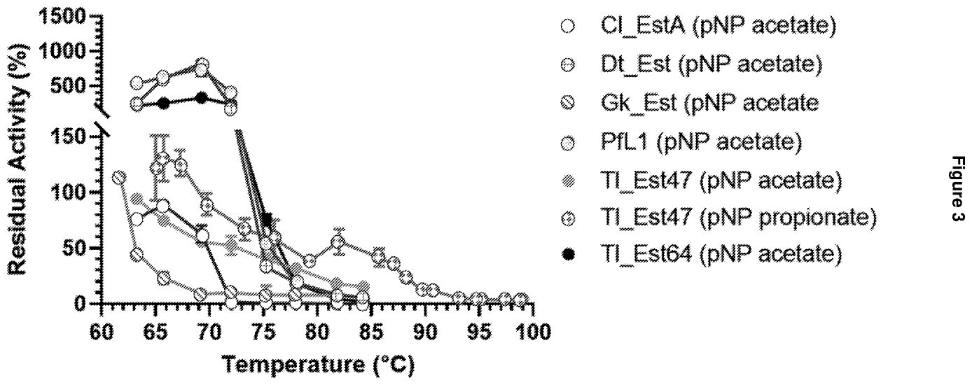

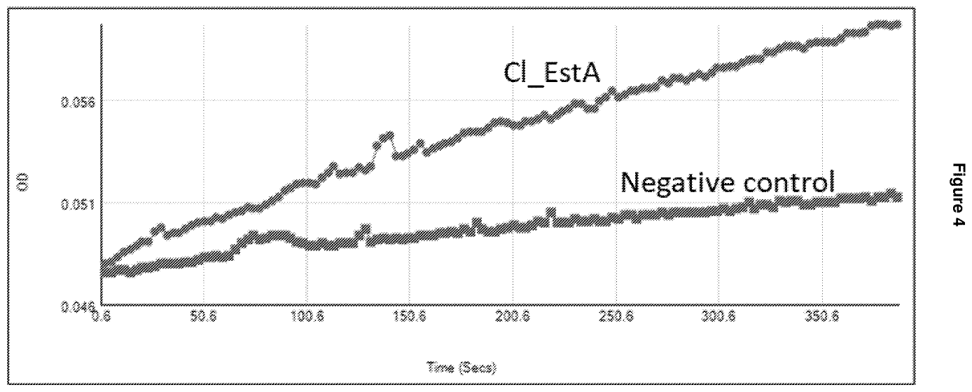

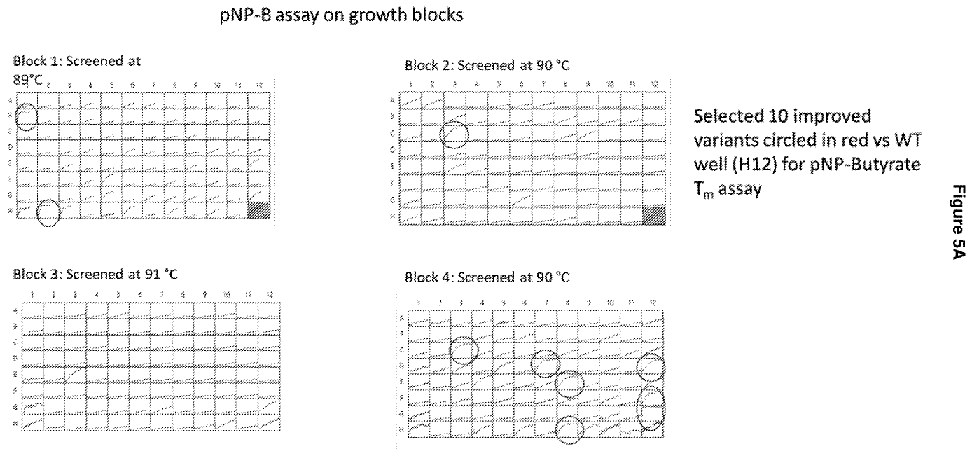

Investigations were then made to determine if the thermostability of Tl_Est47 could be further improved, which retains 10% enzyme activity after being heated at 90° C. for 10 min. To do this, a random mutant library was generated using error prone PCR, with an estimated library size of 13,000 variants with an average of 2-12 mutations per variant. Mutants were cultured overnight in LB media in 96-well growth blocks and subjected to heating at 89, 90 and 91° C. for 10 min with residual activity measured with pNP butyrate (FIG. 4). The 10 mutants with the best activity were then further screened and the mutant P4G12 was observed to have residual activity even after heating for 10 mins at 99° C., which is a ~5-10° C. improvement compared to the WT Tl_Est47 protein (FIG. 5).

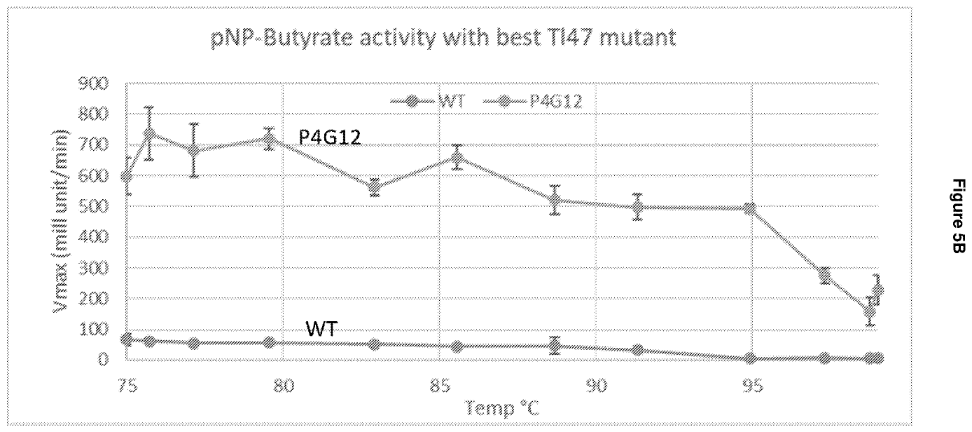

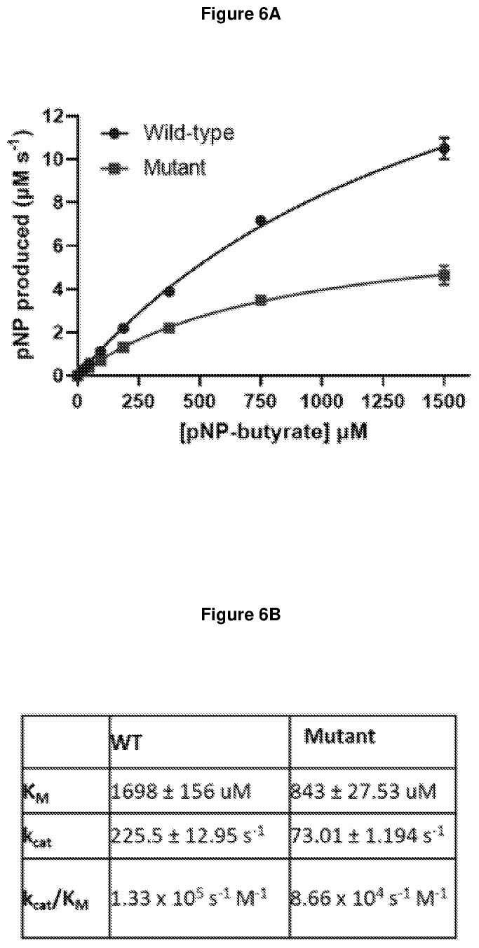

Large scale purification of Tl_Est47 (WT) and the P4G12 mutant was performed to compare the two proteins. Their activities with pNP butyrate (FIGS. 6A and 6B) were first compared, which showed a 3-fold reduction in the turnover rate (k_{cat}) in the mutant compared to the mutant protein. However, the 2-fold decreased Kₘ for the mutant suggests an improved substrate affinity by the mutant, and hence the overall catalytic efficiency (k_{cat}/K_{M}) of the WT is only 1.5-fold higher than the mutant.

Generally, more thermostability is acquired through protein stabilising mutations, that can reduce the overall motion of the protein in solution, reducing protein activity by affecting the rate of substrate binding and diffusion as well as the rate of the overall catalytic mechanism.

The thermostability of the purified WT and mutant proteins was also checked (FIG. 7). It was found that the mutant demonstrated a ~5-7° C. improvement in thermostability when comparing the residual activity after heating in LB media, like the observation in the whole cell assays. The overall thermostability of the WT protein was improved ~5° C. in LB media in comparison to buffer.

### Performance of Variant Tl_Est47 at Differing pH and Temperatures

The performance of the enzyme at a range of pH and temperatures was investigated. The yield of some enzymes can also greatly diminish when transferred to an industrial scale production system (i.e., in a fermenter), increasing the cost of goods (CoG) of the enzyme-based product. Therefore, also investigated was the production of the enzyme by fermentation and comparing it to production in shake flask to determine the enzyme yield under these conditions.

### Methods

### Protein Expression

For protein expression, single colonies from each strain were inoculated into 10 ml of auto-induction media (5 g yeast extract, 20 g tryptone, 85.5 mM NaCl, 22 mM KH₂PO₄, 42 mM Na₂HPO₄, 0.6% glycerol, 0.05% glucose and 0.2% lactose) containing 50 μg/ml of kanamycin (100 μg/mL of ampicillin for the control strain) in 50 ml tubes. Cultures were grown for 3-6 hours at 37° C. while shaking at 200 rpm, followed by overnight incubation at 30° C. The resulting cultures were either stored at 4° C. for up to 2 weeks for whole cell assays or spin down at 4000× g for 10 mins at 4° C., where the supernatant was discarded, and the resulting pellet was stored at −20° C. until protein purification.

Testing for Protein Expression with SDS-PAGE

500 μL from each culture was spun down at 4000× g at 4° C. for 10 mins in 1.5 mL microfuge tubes and the supernatant was discarded. The resulting cell pellet was resuspended in 100 μL of lysis solution (50 mM Tris pH 8, 1×BugBuster Protein Extraction Reagent (Millipore) and ~33 nL DNAse I) and left on ice for about 10 mins. After lysis, 5 μL from each sample was mixed with 10 μL of 50 mM Tris H 8 and 5 μL of 4×NuPAGE™ LDS Sample Buffer (Invitrogen). The rest of the samples were spun down at 20,000×g for 10 mins at 4° C., and 15 μL of the supernatant was mixed with 5 μL of 4×NuPAGE™ LDS Sample Buffer (Invitrogen). The samples were heated at 90° C. for 3 mins and loaded onto pre-cast NuPAGET 4-12% Bis-Tris gels (Invitrogen) and run for 30-40 min at 150 V in MES SDS running buffer (Invitrogen). Gels were stained with AcquaStain Protein Gel Stain (Bulldog) for 30 mins and de-stained in water.

### Protein Purification

For small scale purification, the pellets from 10 mL cultures were resuspended each in 1 ml of lysis buffer containing 50 mM Tris, 300 mM NaCl at pH 8 and transferred to 2 ml microfuge tubes. Cells were lysed by sonication (Fisher Scientific, 5 s pulse, 1 s break, 30 s, 3 times). The lysates were then spun at 20,000×g for 20 min and 4° C. and the supernatant was loaded onto NEBExpress® Ni Spin Columns (New England biolabs) that were pre-washed with 250 μL of the same lysis buffer. The columns were washed with 750 μL total of wash buffer (50 mM Tris, 300 mM NaCl, 5 mM imidazole, pH 8) and the samples were eluted with 2×200 μL of elution buffer (50 mM Tris, 300 mM NaCl, 500 mM imidazole, pH 8). From each eluent, 15 μL was mixed with 5 μL of 4×NuPAGE™ LDS Sample Buffer (Invitrogen) for SDS_PAGE analysis as described above. Purified protein were stored at 4° C. for up to 3 weeks.

### PBSA Degradation Assays

First, 10 mg/mL suspensions of PBSA were prepared in buffer. Then, 250 μL of the suspension was aliquoted into 1.5 mL or 2 mL tubes, one for each enzyme being assayed. For whole cell assays, an additional 250 μL of buffer was added, diluting the PBSA 5 mg/mL, and 5 or 10 μL of cell suspension was added to start the reaction. For the assays with purified enzyme, 250 μL of 200 nM solutions of each enzyme in the same buffer was added to the suspensions, such that the final concentrations of PBSA was 5 mg/ml and enzyme was 100 nM. A negative control with just buffer was also included.

The reactions were incubated, shaking at either room temperature, 37° C. for seven days. For the temperature assays solutions were made using a buffer containing 50 mM Tris pH 8.0. The reactions were incubated at 4, 15, 25, and 37° C. The pH of each stock was adjusted to the desired pH using NaOH as appropriate. Buffer stocks were diluted in distilled water (1 in 10) for the reactions.

At the end of the incubation period, the samples were spun down at 16,000×g for 1 min at room temperature to pellet the remaining plastics, and 2×200 μL aliquots of the supernatant were transferred to a 96-well UV-plate (Grenier) from each tube. To each aliquot 10 μl of 50 mM NAD+ and 5 μl 4U/ml Equine alcohol dehydrogenase was added and the change in absorbance at 340 nm was read at 30 second intervals for 30 min. Values from technical replicates were averaged and 2 or 3 experimental repeats were performed to calculate the standard error from the mean (SEM).

### Comparison of Fermenter and Flask Growth

For a comparison between growth in batch and fermenter culture, cultures were grown in TB or 2YT medium respectively. 2YT Medium (1 L): 5 g yeast extract, 16 g tryptone, 5 g NaCl dissolved in 600 mL distilled water, then made up to 1 L and sterilised at 121° C. for 20 minutes. TB Medium (2.5 L): 12.5 g yeast extract (5 g/L), 50 g tryptone (20 g/L), 12.5 g NaCl (5 g/L), 7.5 g KH₂PO₄ (3 g/L), 14.9 g Na₂HPO₄ (5.96 g/L), 0.6% glycerol (12.5 mL) dissolved in 2.5 L of distilled water then dispensed 500 mL into a sterile, baffled 2 L Erlenmeyer flask with vented cap or 2 L into a fitted Sartorius Biostat B fermenter vessel and sterilised at 121° C. for 60 minutes. After sterilisation kanamycin was added at a final concentration of 50 mg/mL.

The 500 mL flask and 2 L fermenter were inoculated from 10 mL overnight cultures in the same media that had been seeded with a single colony from an agar plate streaked with E. coli BL21 DE3 transformed with the appropriate expression plasmid.

The fermenter control parameters were: pO₂ setpoint=30%, cascade: stirrer/airflow/O₂ enrich, Initial setpoint: stirrer=500 rpm, airflow=0.3 L/min, 02=0.07 L/min, temperature setpoint=37° C., pH setpoint=7.0, acid/base configured, 10% H₃PO₄/10% NH₃.

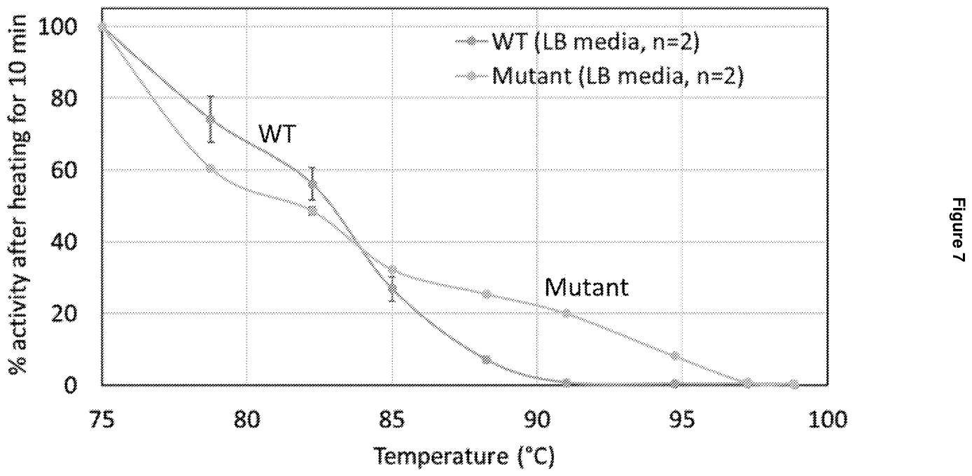

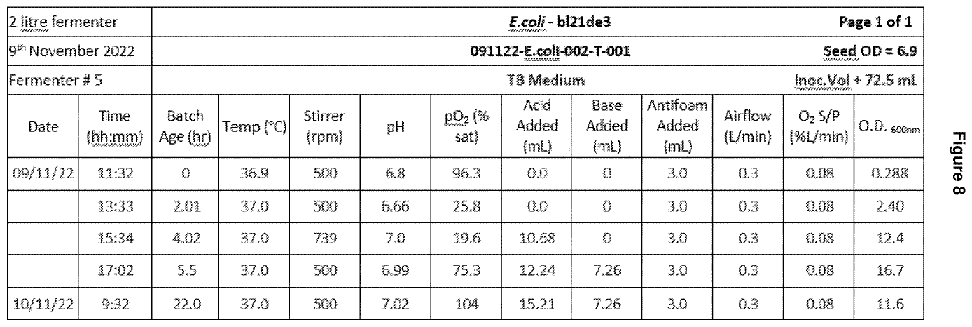

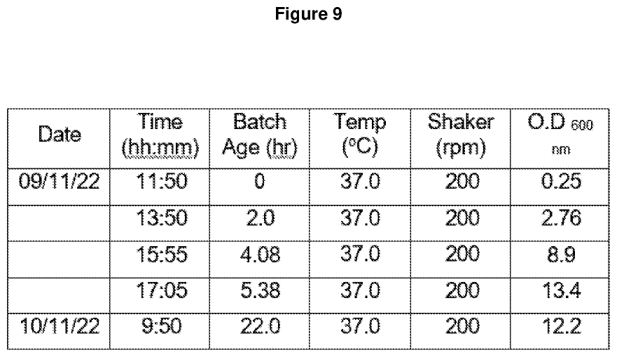

Samples to measure OD and 1 mL samples for enzyme analysis were taken throughout the process. At harvest, a 20 mL cell pellet, and the total harvest pellet, weighing 36 g, were retained and frozen at −80, together with the 1 mL samples. Logs of the conditions in the flask and fermenter are given in FIGS. 8 and 9.

### Performance of Variant TI Est47 at Differing pH and Temperatures

Tl_Est47 and its variant were purified from E. coli BL21 DE3 cultures and used in this study. The purified proteins were incubated for seven days with PBSA, with estimates of alcohol dehydrogenase activity taken every day.

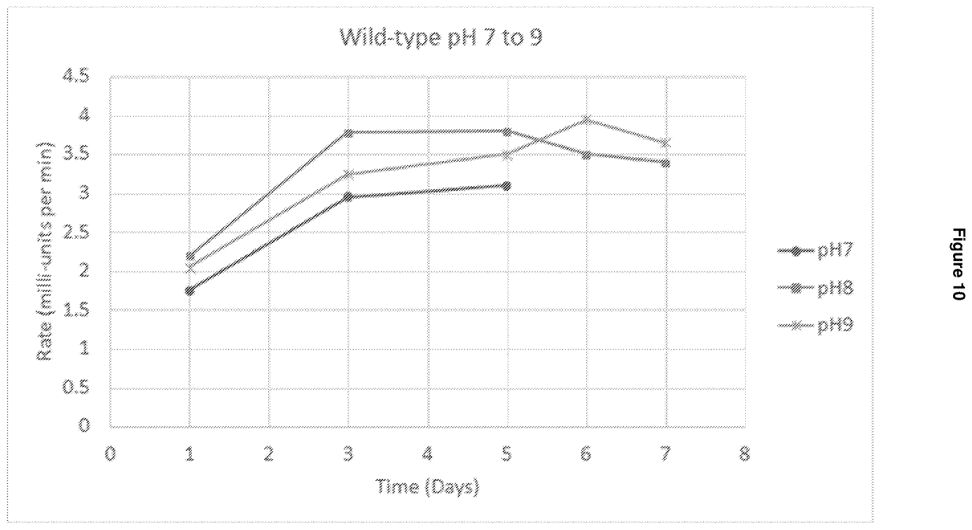

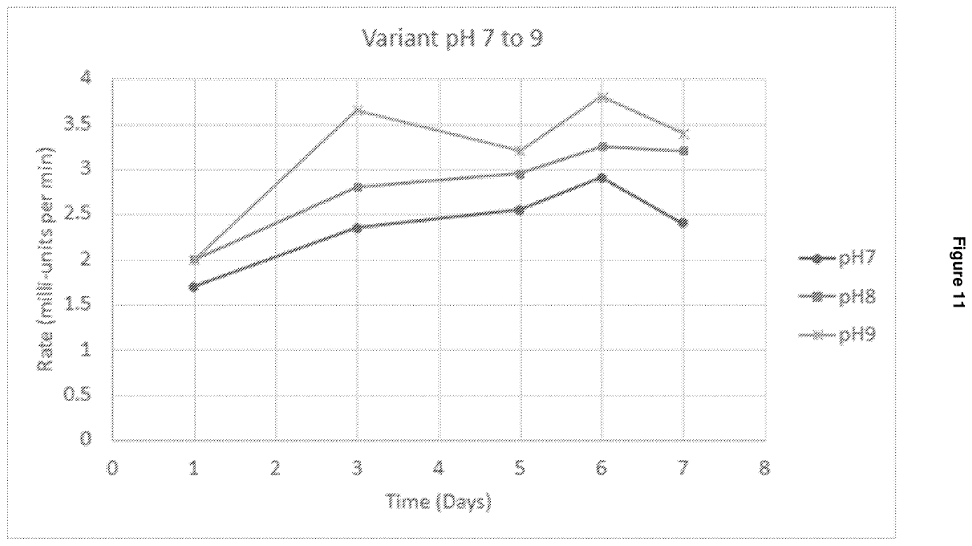

Both the wild-type and variant responded to pH as expected (FIGS. 10 & 11). Serine hydrolases tend to have a pH optimum above pH 8, due to the pKa of the serine nucleophile in the active site. At lower pH values the activity drops.

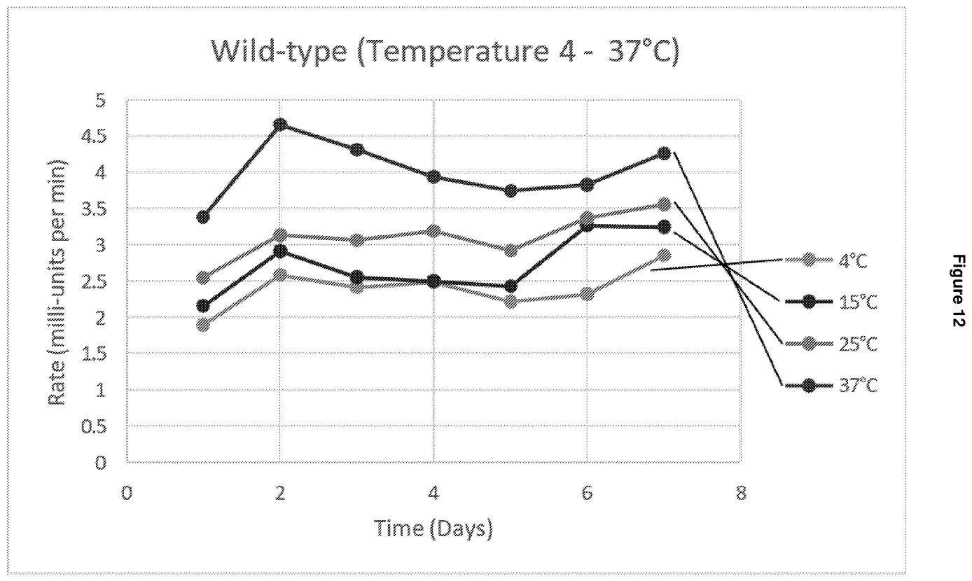

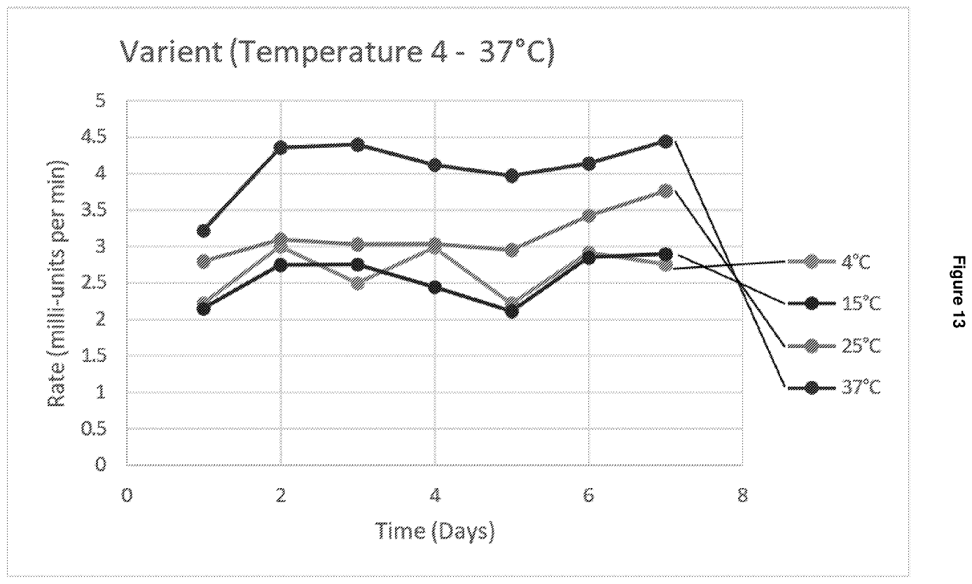

The variant and wild-type enzymes had similar activities to each other at temperatures between 4 and 37° C., showing that the amino acid substitutions in the variant didn't affect the activity of the enzyme at low temperature and that the enzymes are active at temperatures likely to reflect those found during its application (FIGS. 12 & 13). As expected, the activity of both enzymes increases as temperature does, with activity approximately doubled when incubated at 37° C. compared to 4° C.

### Production of TI Est47 by Fermentation

E. coli BL21 DE3 expressing the Tl_Est47 variant was grown in a 500 mL flask culture and a 2 L fermenters to estimate the production of Tl_Est47 in both conditions. enzyme production capacity in fermenters (2 L scale). The two culture systems were compared with 1 mL of culture sampled and flash-frozen in liquid nitrogen at −80° C. over the course of the expression at 2, 4 5.5 and 22 hours. The total amount of protein in each 1 ml sample was estimated (Table 1). After 5.5 hours the total protein plateaued. After 22 hours of expression the cultures were harvested with 4.9 g of protein from the flask incubation and 22 g from the fermenter.

TABLE 1
Estimation of total amount of protein produced during
incubation of 500 mL flask and 2 L fermenter.

| | | Total protein | Total |
|---|---|---|---|
| | Time | concentration | protein |
| | (hours) | (mg/mL) | amount (g) |
| Flask | 2 | 8.2 | 4.25 |
| | 4 | 8.6 | 4.3 |
| | 5.5 | 10.9 | 5.45 |
| | 22 | 9.8 | 4.9 |
| Reactor | 2 | 9.4 | 18.8 |
| | 4 | 9.6 | 19.2 |
| | 5.5 | 10.5 | 21 |
| | 22 | 11.0 | 22 |

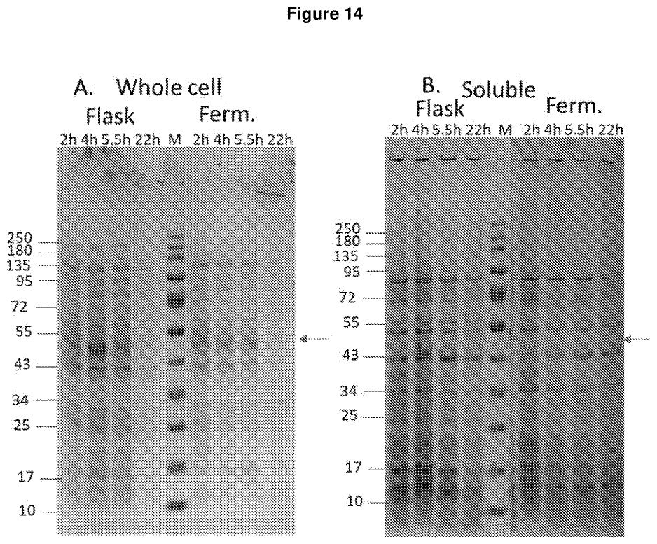

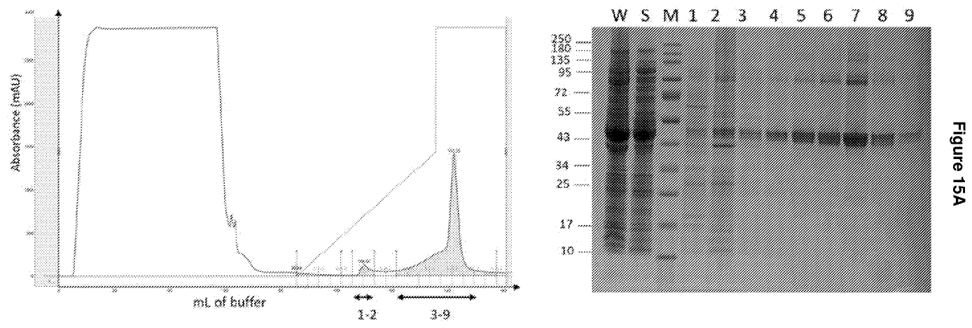

After thawing on ice, samples were sonicated for 30 seconds and whole cell and soluble protein was analysed by SDS/PAGE (FIG. 14).

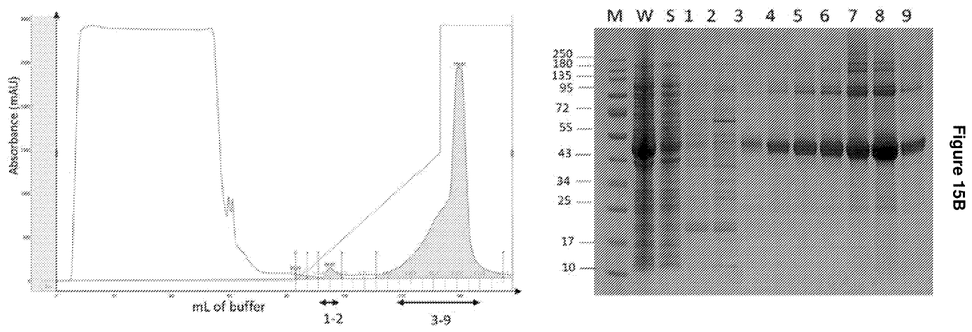

After 22 hours of flask cultivation or fermentation, pellets were harvested 6.5 g of pellet were obtained for the 500 mL flask and 36 g for the 2 L fermenter. We used 6.5 g and 6 g of the flask's pellet and of the fermenter's pellet and purified the protein by FPLC using a His-trap column on an AKTA pure FPLC. Analysis of fractions by SDS-PAGE and traces are shown in FIG. 15A/B.

Fractions 3-9 for each of the systems were pooled and concentrated on Amicon column cutoff 10 kDa, followed by buffer exchange to remove residual imidazole. Protein concentration was estimated by Nanodrop (extinction coefficient at 280 nM: 124,470). Enzyme purified from the flask produced 14 mL of purified enzyme at 0.9 mg/mL, from the 6.5 g pellet processed. The enzyme produced in the fermenter gave 16 mL of purified enzyme at 2.6 mg/mL from the 6 g of pellet processed. A summary of the amount of enzyme produced by our two-systems can be found in Table 2.

TABLE 2
Purification summary.

| | Total | Amount of enzyme | Total amount | Percentage of |
|---|---|---|---|---|
| | amount | per gram of pellet | of enzyme | enzyme in the |
| | of protein (g) | (mg/g) | produced (mg) | total protein |
| Flask | 4.9 | 1.9 | 12.6 | 0.3% |
| Fermenter | 22 | 6.9 | 249.6 | 1.1% |

The protein yields were good and production of the Tl_Est47 variant in the fermenter improved both the proportion and amount compared to the same protein produced in shake flasks. However, in both cases the percentage of Tl_Est47 variant produced was lower than observed for some highly produced heterologously expressed proteins, suggesting that there is scope to improve the expression level and production yield of the Tl_Est47 variant if desired. This will ultimately reduce the cost of goods for enzyme production and the final product. A further 10 L fermentation was run (estimated Tl_Est47 variant yield ~1.2 g), and the cell pellet stored at −70° C.

The engineering used to generate the variant of Tl_Est47 with increased thermal stability did not negatively affect the stability or activity of the protein at a range of temperatures or pH values. The Tl_Est47 maintained substantial activity at low temperature, suggesting that it will retain activity under conditions likely to be encountered in use. The heterologous production of the Tl_Est47 variant was substantially improved when conducted in a fermenter compared to a shake flask. Although the expression levels were good, the yields of protein could potentially be improved further to reduce the cost of production.

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, nucleic acid sequences are written left to right in 5′ to 3′ orientation; and amino acid sequences are written left to right in amino to carboxy orientation.

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T.A. Brown (editor), Essential Molecular Biology: A Practical Approach, Volumes 1 and 2, IRL Press (1991), D.M. Glover and B.D. Hames (editors), DNA Cloning: A Practical Approach, Volumes 1-4, IRL Press (1995 and 1996), and F.M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J.E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning. Any definitions provided herein are to be interpreted in the context of the specification as a whole. As used herein, the singular “a”, “an”, “said”, and “the” includes the plural unless the context clearly indicates otherwise. For example, reference to “a protein” includes a plurality of proteins, unless the context clearly is to the contrary. As used herein, the term “protein” includes proteins, polypeptides, and peptides. In some embodiments, the terms “protein”, “polypeptide” and “peptide” can be used interchangeably.

As used herein, the term about, unless stated to the contrary, refers to +/−20%, more preferably +/−10%, even more preferably +/−5%, of the designated value. Each numerical range used herein includes every narrower numerical range that falls within such broader numerical range, as if such narrower numerical ranges were all expressly written herein.

For the purposes of this specification and appended claims, the term “about” or “substantially” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The terms “polypeptide” and “protein” are generally used interchangeably and refer to a single polypeptide chain (polymeric sequence of amino acid residues) which may or may not be modified by addition of non-amino acid groups. It would be understood that such polypeptide chains may associate with other polypeptides or proteins or other molecules such as co-factors, The terms “proteins” and “polypeptides” as used herein also include variants, mutants, biologically active fragments, and/or modifications of the polypeptides described herein. The single and 3-letter code for amino acids as defined in conformity with the IUPAC-IUB Joint Commission on Biochemical Nomenclature (JCBN) is used throughout this disclosure. A single letter X refers to any of the twenty amino acids. It is also understood that a polypeptide may be coded for by more than one nucleotide sequence due to the degeneracy of the genetic code.

The % identity of a polypeptide may be determined by GAP (Needleman and Wunsch, 1970) analysis (GCG program) with a gap creation penalty=5, and a gap extension penalty=0.3. The query sequence is at least 250 amino acids in length and the GAP analysis aligns the two sequences over a region of at least 250 amino acids. More preferably, the query sequence is at least 300 amino acids in length and the GAP analysis aligns the two sequences over a region of at least 300 amino acids. Even more preferably, the query sequence is at least 350 amino acids in length and the GAP analysis aligns the two sequences over a region of at least 350 amino acids. Even more preferably, the GAP analysis aligns the two sequences over their entire length.

As used herein, the phrase “at a position corresponding to amino acid number” refers to the relative position of the amino acid compared to surrounding amino acids with reference to a defined amino acid sequence. For instance, in some embodiments a polypeptide of the invention may have additional N-terminal amino acids to assist with intracellular localization or extracellular secretion which alters the relative positioning of the amino acid when aligned against, for example, SEQ ID NO:3 or SEQ ID NO:4.

The term “mature” form of a protein, polypeptide, or peptide refers to the functional form of the protein, polypeptide, or peptide without the signal peptide sequence and propeptide sequence.

As used herein with regard to amino acid residue positions, “corresponding to” or “corresponds to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related proteins or a reference protein.

The term “wild-type” in reference to an amino acid sequence or nucleic acid sequence indicates that the amino acid sequence or nucleic acid sequence is a native or naturally-occurring sequence. As used herein, the term “naturally-occurring” refers to anything (e.g., protein or polynucleotide sequences) that is found in nature. Conversely, the term “non-naturally occurring” refers to anything that is not found in nature (e.g., recombinant polynucleotide and protein sequences produced in the laboratory or modification of the wild-type sequence).

The term “improved thermostability” or “enhanced thermostability” or “increased thermostability” as used herein refers to a novel polypeptide displaying increased retention of enzyme activity after a period of incubation at elevated temperatures, particularly with respect to the wild-type of similar enzymes or polypeptides. In addition, the terms “improved thermostable properties” and “thermostability” are herein used interchangeably for the purposes of the specification and claims.

The term “variant,” with respect to a polypeptide amino acid sequence, refers to a polypeptide amino acid sequence that differs from a specified wild-type, parental, or reference polypeptide amino acid sequence by including one or more man-made substitutions, insertions, or deletions of an amino acid. Similarly, the term “variant,” with respect to a polynucleotide nucleic acid sequence, refers to a polynucleotide nucleic acid sequence that differs from a specified wild-type, parental, or reference polynucleotide by including one or more man-made substitutions, insertions, or deletions of a nucleic acid. The identity of the wild-type, parental, or reference polypeptide amino acid sequence or polynucleotide nucleic acid sequence will be apparent from context.

As used herein, the term “mutation” or “engineering” refers to man-made changes to a reference amino acid or nucleic acid sequence. It is intended that the term encompass man-made substitutions, insertions and deletions.

As used herein, the term “vector” refers to a nucleic acid construct used to introduce or transfer nucleic acid(s) into a target cell or tissue. A vector is typically used to introduce foreign DNA into a cell or tissue. Vectors include plasmids, cloning vectors, bacteriophages, viruses (e.g., viral vector), cosmids, expression vectors, shuttle vectors, and the like. A vector typically includes an origin of replication, a multicloning site, and a selectable marker. The process of inserting a vector into a target cell is typically referred to as transformation.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, electroporation, conjugation, and transduction. Transformation refers to the genetic alteration of a cell which results from the uptake, optional genomic incorporation, and expression of genetic material (e.g., DNA).

“Expression cassette” or “expression vector” refers to a nucleic acid construct or vector generated recombinantly or synthetically for the expression of a nucleic acid of interest (e.g., a foreign nucleic acid or transgene) in a target cell. The nucleic acid of interest typically expresses a protein of interest. An expression vector or expression cassette typically comprises a promoter nucleotide sequence that drives or promotes expression of the foreign nucleic acid. The expression vector or cassette also typically includes other specified nucleic acid elements that permit transcription of a particular nucleic acid in a target cell. A recombinant expression cassette can be incorporated into a plasmid, chromosome, mitochondrial DNA, plastid DNA, virus, or nucleic acid fragment. Some expression vectors have the ability to incorporate and express heterologous DNA fragments in a host cell or genome of the host cell. Many prokaryotic and eukaryotic expression vectors are commercially available. Selection of appropriate expression vectors for expression of a protein from a nucleic acid sequence incorporated into the expression vector is within the knowledge of those of skill in the art.

As used herein, a nucleic acid is “operably linked” with another nucleic acid sequence when it is placed into a functional relationship with another nucleic acid sequence. For example, a promoter or enhancer is operably linked to a nucleotide coding sequence if the promoter affects the transcription of the coding sequence. A ribosome binding site may be operably linked to a coding sequence if it is positioned so as to facilitate translation of the coding sequence. Typically, “operably linked” DNA sequences are contiguous. However, enhancers do not have to be contiguous. Linking is accomplished by ligation at convenient restriction sites. If such sites do not exist, synthetic oligonucleotide adaptors or linkers may be used in accordance with conventional practice.

As used herein the term “gene” refers to a polynucleotide (e.g., a DNA segment), that encodes a polypeptide and includes regions preceding and following the coding regions. In some instances a gene includes intervening sequences (introns) between individual coding segments (exons).

As used herein, “recombinant” when used with reference to a cell typically indicates that the cell has been modified by the introduction of a foreign nucleic acid sequence or that the cell is derived from a cell so modified. For example, a recombinant cell may comprise a gene not found in identical form within the native (non-recombinant) form of the cell, or a recombinant cell may comprise a native gene (found in the native form of the cell) that has been modified and re-introduced into the cell. A recombinant cell may comprise a nucleic acid endogenous to the cell that has been modified without removing the nucleic acid from the cell; such modifications include those obtained by gene replacement, site-specific mutation, and related techniques known to those of ordinary skill in the art. Recombinant DNA technology includes techniques for the production of recombinant DNA in vitro and transfer of the recombinant DNA into cells where it may be expressed or propagated, thereby producing a recombinant polypeptide. “Recombination” and “recombining” of polynucleotides or nucleic acids refer generally to the assembly or combining of two or more nucleic acid or polynucleotide strands or fragments to generate a new polynucleotide or nucleic acid.

A nucleic acid or polynucleotide is said to “encode” a polypeptide if, in its native state or when manipulated by methods known to those of skill in the art, it can be transcribed and/or translated to produce the polypeptide or a fragment thereof. The anti-sense strand of such a nucleic acid is also said to encode the sequence.

The terms “host strain” and “host cell” refer to a suitable host for an expression vector comprising a DNA sequence of interest.

The term “precursor” form of a protein or peptide refers to a mature form of the protein having a prosequence operably linked to the amino or carbonyl terminus of the protein. The precursor may also have a “signal” sequence operably linked to the amino terminus of the prosequence. The precursor may also have additional polypeptides that are involved in post-translational activity (e.g., polypeptides cleaved therefrom to leave the mature form of a protein or peptide).

The terms “derived from” and “obtained from” refer to not only a protein produced or producible by a strain of the organism in question, but also a protein encoded by a DNA sequence isolated from such strain and produced in a host organism containing such DNA sequence. Additionally, the term refers to a protein which is encoded by a DNA sequence of synthetic and/or cDNA origin and which has the identifying characteristics of the protein in question.

The term “identical” in the context of two polynucleotide or polypeptide sequences refers to the nucleic acids or amino acids in the two sequences that are the same when aligned for maximum correspondence, as measured using sequence comparison or analysis algorithms described below and known in the art.

“% identity” or percent identity” or “PID” refers to protein sequence identity. Percent identity may be determined using standard techniques known in the art. The percent amino acid identity shared by sequences of interest can be determined by aligning the sequences to directly compare the sequence information, e.g., by using a program such as BLAST, MUSCLE, or CLUSTAL. The BLAST algorithm is described, for example, in Altschul et al., J Mol Biol, 215:403-410 (1990) and Karlin et al., Proc Natl Acad Sci USA, 90:5873-5787 (1993). A percent (%) amino acid sequence identity value is determined by the number of matching identical residues divided by the total number of residues of the “reference” sequence including any gaps created by the program for optimal/maximum alignment. BLAST algorithms refer to the “reference” sequence as the “query” sequence.

The CLUSTAL W algorithm is another example of a sequence alignment algorithm (See, Thompson et al., Nucleic Acids Res, 22:4673-4680, 1994). Default parameters for the CLUSTAL W algorithm include: Gap opening penalty=10.0; Gap extension penalty=0.05; Protein weight matrix=BLOSUM series; DNA weight matrix=IUB; Delay divergent sequences %=40; Gap separation distance=8; DNA transitions weight=0.50; List hydrophilic residues=GPSNDQEKR; Use negative matrix=OFF; Toggle Residue specific penalties=ON; Toggle hydrophilic penalties=ON; and Toggle end gap separation penalty=OFF. In CLUSTAL algorithms, deletions occurring at either terminus are included. For example, a variant with a five amino acid deletion at either terminus (or within the polypeptide) of a polypeptide of 500 amino acids would have a percent sequence identity of 99% (495/500 identical residues x 100) relative to the “reference” polypeptide. Such a variant would be encompassed by a variant having “at least 99% sequence identity” to the polypeptide.

Understanding the homology between molecules can reveal the evolutionary history of the molecules as well as information about their function; if a newly sequenced protein is homologous to an already characterized protein, there is a strong indication of the new protein's biochemical function. The most fundamental relationship between two entities is homology; two molecules are said to be homologous if they have been derived from a common ancestor. Homologous molecules, or homologs, can be divided into two classes, paralogs and orthologs. Paralogs are homologs that are present within one species. Paralogs often differ in their detailed biochemical functions. Orthologs are homologs that are present within different species and have very similar or identical functions. A protein superfamily is the largest grouping (clade) of proteins for which common ancestry can be inferred. Usually this common ancestry is based on sequence alignment and mechanistic similarity. Superfamilies typically contain several protein families which show sequence similarity within the family. The term “protein clan” is commonly used for protease superfamilies based on the MEROPS protease classification system.

A nucleic acid or polynucleotide is “isolated” when it is at least partially or completely separated from other components, including but not limited to for example, other proteins, nucleic acids, cells, etc. Similarly, a polypeptide, protein or peptide is “isolated” when it is at least partially or completely separated from other components, including but not limited to for example, other proteins, nucleic acids, cells, etc. On a molar basis, an isolated species is more abundant than are other species in a composition. For example, an isolated species may comprise at least about 60%, about 65%, about 70%, about 75%, about 80%, about 85%, about 90%, about 91%, about 92%, about 93%, about 94%, about 95%, about 96%, about 97%, about 98%, about 99%, or about 100% (on a molar basis) of all macromolecular species present. Preferably, the species of interest is purified to essential homogeneity (i.e., contaminant species cannot be detected in the composition by conventional detection methods). Purity and homogeneity can be determined using a number of techniques well known in the art, such as agarose or polyacrylamide gel electrophoresis of a nucleic acid or a protein sample, respectively, followed by visualization upon staining. If desired, a high-resolution technique, such as high performance liquid chromatography (HPLC) or a similar means can be utilized for purification of the material.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

One or more novel polypeptide variant described herein can be subject to various changes, such as one or more amino acid insertion, deletion, and/or substitution, either conservative or non-conservative, including where such changes do not substantially alter the enzymatic activity of the variant. Similarly, a nucleic acid of the invention can also be subject to various changes, such as one or more substitution of one or more nucleotide in one or more codon such that a particular codon encodes the same or a different amino acid, resulting in either a silent variation (e.g., when the encoded amino acid is not altered by the nucleotide mutation) or non-silent variation; one or more deletion of one or more nucleic acids (or codon) in the sequence; one or more addition or insertion of one or more nucleic acids (or codon) in the sequence; and/or cleavage of, or one or more truncation, of one or more nucleic acid (or codon) in the sequence. Many such changes in the nucleic acid sequence may not substantially alter the enzymatic activity of the resulting encoded polypeptide enzyme compared to the polypeptide enzyme encoded by the original nucleic acid sequence. A nucleic acid sequence described herein can also be modified to include one or more codon that provides for optimum expression in an expression system (e.g., bacterial expression system), while, if desired, said one or more codon still encodes the same amino acid(s).

One or more nucleic acid sequence described herein can be generated by using any suitable synthesis, manipulation, and/or isolation techniques, or combinations thereof. For example, one or more polynucleotide described herein may be produced using standard nucleic acid synthesis techniques, such as solid-phase synthesis techniques that are well-known to those skilled in the art. In such techniques, fragments of up to 50 or more nucleotide bases are typically synthesized, then joined (e.g., by enzymatic or chemical ligation methods) to form essentially any desired continuous nucleic acid sequence. The synthesis of the one or more polynucleotide described herein can be also facilitated by any suitable method known in the art, including but not limited to chemical synthesis using the classical phosphoramidite method (See e.g., Beaucage et al. Tetrahedron Letters 22:1859-69 (1981)), or the method described in Matthes et al., EMBO J.3:801-805 (1984) as is typically practiced in automated synthetic methods. One or more polynucleotide described herein can also be produced by using an automatic DNA synthesizer. Customized nucleic acids can be ordered from a variety of commercial sources (e.g., Midland Certified Reagent Company, Great American Gene Company, Operon Technologies Inc., and DNA 2.0). Other techniques for synthesizing nucleic acids and related principles are described by, for example, Itakura et al., Ann. Rev. Biochem.53:323 (1984) and Itakura et al., Science 198:1056 (1984).

A further embodiment is directed to one or more vector comprising one or more novel polypeptide variant described herein (e.g., a polynucleotide encoding one or more novel polypeptide variant described herein); expression vectors or expression cassettes comprising one or more nucleic acid or polynucleotide sequence described herein; isolated, substantially pure, or recombinant DNA constructs comprising one or more nucleic acid or polynucleotide sequence described herein; isolated or recombinant cells comprising one or more polynucleotide sequence described herein; and compositions comprising one or more such vector, nucleic acid, expression vector, expression cassette, DNA construct, cell, cell culture, or any combination or mixtures thereof.

Some embodiments are directed to one or more recombinant cell comprising one or more vector (e.g., expression vector or DNA construct) described herein which comprises one or more nucleic acid or polynucleotide sequence described herein. Some such recombinant cells are transformed or transfected with such at least one vector, although other methods are available and known in the art. Such cells are typically referred to as host cells. Some such cells comprise bacterial cells. Other embodiments are directed to recombinant cells (e.g., recombinant host cells) comprising one or more novel polypeptide described herein.

In some embodiments, one or more vector described herein is an expression vector or expression cassette comprising one or more polynucleotide sequence described herein operably linked to one or more additional nucleic acid segments required for efficient gene expression (e.g., a promoter operably linked to one or more polynucleotide sequence described herein). A vector may include a transcription terminator and/or a selection gene (e.g., an antibiotic resistant gene) that enables continuous cultural maintenance of plasmid-infected host cells by growth in antimicrobial-containing media. An expression vector may be derived from plasmid or viral DNA, or in alternative embodiments, contains elements of both.

For expression and production of a protein of interest (e.g., one or more novel polypeptide described herein) in a cell, one or more expression vector comprising one or more copy of a polynucleotide encoding one or more novel polypeptide described herein, and in some instances comprising multiple copies, is transformed into the cell under conditions suitable for expression of the novel polypeptide. In some embodiments, a polynucleotide sequence encoding one or more novel polypeptide described herein (as well as other sequences included in the vector) is integrated into the genome of the host cell, while in other embodiments, a plasmid vector comprising a polynucleotide sequence encoding one or more novel polypeptide described herein remains as autonomous extra-chromosomal element within the cell. Some embodiments provide both extrachromosomal nucleic acid elements as well as incoming nucleotide sequences that are integrated into the host cell genome. The vectors described herein are useful for production of the novel polypeptides described herein. In some embodiments, a polynucleotide construct encoding one or more novel polypeptide described herein is present on an integrating vector that enables the integration and optionally the amplification of a polynucleotide encoding a novel polypeptide into the host chromosome. Examples of sites for integration are well known to those skilled in the art. In some embodiments, transcription of a polynucleotide encoding a novel polypeptide described herein is effectuated by a promoter that is the wild-type promoter for the wild-type polypeptide. In some other embodiments, the promoter is heterologous to the one or more novel polypeptide described herein, but is functional in the host cell.

In addition to commonly used methods, in some embodiments, host cells are directly transformed with a DNA construct or vector comprising a nucleic acid encoding one or more novel polypeptides described herein (i.e., an intermediate cell is not used to amplify, or otherwise process, the DNA construct or vector prior to introduction into the host cell). Introduction of a DNA construct or vector described herein into the host cell includes those physical and chemical methods known in the art to introduce a nucleic acid sequence (e.g., DNA sequence) into a host cell without insertion into the host genome. Such methods include, but are not limited to calcium chloride precipitation, electroporation, naked DNA, and liposomes. In additional embodiments, DNA constructs or vector are co-transformed with a plasmid, without being inserted into the plasmid. In further embodiments, a selective marker is deleted from the altered bacterial strain by methods known in the art (See, Stahl et al., J. Bacteriol. 158:411-418 (1984); and Palmeros et al., Gene 247:255-264 (2000)). In some embodiments, the transformed cells are cultured in conventional nutrient media. The suitable specific culture conditions, such as temperature, pH and the like are known to those skilled in the art and are well described in the scientific literature.

As used herein, the term “extract” refers to any portion of a host cell or non-human transgenic organism of the invention comprising a polypeptide of the invention, preferably also comprising a polynucleotide or vector of the invention. This term includes portions secreted from the host cell, and hence encompasses culture supernatants. Preferably the extract is a relatively crude extract which has not undergone a purification step to purify the polypeptide of the invention away from other polypeptides which were co-produced with the polypeptide of the invention. An extract may also be a composition comprising a polypeptide of the invention.

As used herein a “biologically active fragment” is a portion of a polypeptide as described herein which maintains a defined activity of the full-length polypeptide. Biologically active fragments can be any size as long as they maintain the defined activity.

Thus, where applicable, in light of the minimum % identity figures, it is preferred that the polypeptide comprises an amino acid sequence which is at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO. With regard to a defined polypeptide, it will be appreciated that % identity figures higher than those provided herein will encompass preferred embodiments.

By “substantially purified” or “purified” we mean a polypeptide that has been separated from one or more lipids, nucleic acids, other polypeptides, or other contaminating molecules with which it is associated in its native state. It is preferred that the substantially purified polypeptide is at least 60% free, more preferably at least 75% free, and more preferably at least 90% free from other components with which it is naturally associated. Whilst at present there is no evidence that the polypeptides of the invention exist in nature, the terms native state and naturally associated also encompass the polypeptide produced in a host cell of the invention.

The term “recombinant” in the context of a polypeptide refers to the polypeptide when produced by a cell, or in a cell-free expression system, in an altered amount or at an altered rate compared to its native state. In one embodiment, the cell is a cell that does not naturally produce the polypeptide. A recombinant polypeptide of the invention includes polypeptides which have not been separated from other components of the transgenic (recombinant) cell, or cell-free expression system, in which it is produced, and polypeptides produced in such cells or cell-free systems which are subsequently purified away from at least some other components.

Amino acid sequence mutants or variants of a polypeptide described herein can be prepared by introducing appropriate nucleotide changes into a nucleic acid defined herein, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics.

Mutant or variant polypeptides can be prepared using any technique known in the art, for example, using directed evolution or rational design strategies (see below). Products derived from mutated/altered DNA can readily be screened using techniques described herein to determine if they possess enzyme activity.

In designing amino acid sequence variants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. Sites of interest are those in which particular residues obtained from various strains or species are identical. These positions may be important for biological activity. These sites, especially those falling within a sequence of at least three other identically conserved sites, are preferably substituted in a relatively conservative manner.

In a preferred embodiment, a mutant/variant polypeptide has only conservative substitutions when compared to a novel polypeptide specifically defined herein. In a preferred embodiment a mutant/variant polypeptide has one or two or three or four conservative amino acid changes when compared to a novel polypeptide specifically defined herein.

Preferably, if not specified otherwise, at a given amino acid position the novel polypeptide comprises an amino acid as found at the corresponding position of the polypeptide provided as SEQ ID NO: 4.

Also included within the scope of the invention are novel polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide.

Novel polypeptides described herein can be produced in a variety of ways, including production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

In an embodiment, a novel polypeptide of the invention comprises a signal sequence which is capable of directing secretion of the polypeptide from a cell. As the skilled person would appreciate, the signal sequence may or not be cleaved, or be partially cleaved, whilst being partially exported from the cell. However, when removing a signal sequence the cell may produce a heterogeneous population of polypeptides with slightly different, for example, N-terminal sequences. Thus, the term “consists of encompasses such variants produced by the removal of signal sequences. A large number of such signal sequences have been isolated, which include N- and C-terminal signal sequences. Prokaryotic and eukaryotic N-terminal signal sequences are similar, and it has been shown that eukaryotic N-terminal signal sequences are capable of functioning as secretion sequences in bacteria. An example of such an N-terminal signal sequence is the bacterial B-lactamase signal sequence, which is a well-studied sequence, and has been widely used to facilitate the secretion of polypeptides into the external environment. An example of C-terminal-signal sequences is the hemolysin A (hlyA) signal sequences of E. coli. Additional examples of signal sequences include, without limitation, aerolysin, alkaline phosphatase gene (phoA), chitinase, endochitinase, a-hemolysin, MlpB, pullulanase, Yops and a TAT signal peptide.

As used herein, an “isolated polynucleotide” means a polynucleotide which is at least partially separated from the polynucleotide sequences with which it is associated or linked in its native state. Isolated polynucleotides include DNA and RNA molecules, and molecules that are a combination of DNA and RNA. They may be single-stranded, double stranded or partially double-stranded, and may be in a sense or antisense orientation with respect to a promoter. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid”.

The term “exogenous” in the context of a polynucleotide refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered, preferably increased, amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components.

The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns the two sequences over their entire length.

Polynucleotides of the present invention may possess, when compared to molecules provided herewith, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

Usually, monomers of a polynucleotide are linked by phosphodiester bonds or analogs thereof. Analogs of phosphodiester linkages include: phosphorothioate, phosphorodithioate, phosphoroselenoate, phosphorodiselenoate, phosphoroanilothioate, phosphoranilidate and phosphoramidate.

One embodiment of the present invention includes a recombinant vector, which comprises at least one isolated/exogenous polynucleotide of the invention inserted into any vector capable of delivering the polynucleotide molecule into a host cell. Such a vector contains heterologous polynucleotide sequences, that is polynucleotide sequences that are not naturally found adjacent to polynucleotide molecules of the present invention and that preferably are derived from a species other than the species from which the polynucleotide molecule(s) are derived. The vector can be either RNA or DNA, either prokaryotic or eukaryotic, and typically is a transposon, a virus or a plasmid.

One type of recombinant vector comprises the polynucleotide(s) operably linked to an expression vector. The phrase operably linked refers to insertion of a polynucleotide molecule into an expression vector in a manner such that the molecule is able to be expressed when transformed into a host cell. As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Expression vectors include any vectors that function (i.e., direct gene expression) in recombinant cells, including in bacterial, fungal, endoparasite, arthropod, animal, and plant cells. Vectors of the invention can also be used to produce the polypeptide in a cell-free expression system, such systems are well known in the art.

“Operably linked” as used herein refers to a functional relationship between two or more nucleic acid (e.g., DNA) segments. Typically, it refers to the functional relationship of transcriptional regulatory element to a transcribed sequence. For example, a promoter is operably linked to a coding sequence, such as a polynucleotide defined herein, if it stimulates or modulates the transcription of the coding sequence in an appropriate host cell and/or in a cell-free expression system. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

In particular, expression vectors according to the present invention contain regulatory sequences such as transcription control sequences, translation control sequences, origins of replication, and other regulatory sequences that are compatible with the recombinant cell and that control the expression of polynucleotide molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod, nematode, plant or animal cells, such as, but not limited to, tac, lac, tip, trc, oxy-pro, omp/lpp, rmB, bacteriophage lambda, bacteriophage T7, T71ac, bacteriophage T3, bacteriophage SP6, bacteriophage SP01, metallothionein, alpha-mating factor, Pichia alcohol oxidase, alpha virus subgenomic promoters (such as Sindbis virus subgenomic promoters), antibiotic resistance gene, baculovirus, Heliothis tea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells.

Another embodiment of the present invention includes a host cell transformed with one or more recombinant molecules described herein or progeny cells thereof. Transformation of a polynucleotide molecule into a cell can be accomplished by any method by which a polynucleotide molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotide molecules of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide of the present invention. Host cells of the present invention either can be endogenously (i.e., naturally) capable of producing polypeptides described herein or can be capable of producing such polypeptides after being transformed with at least one polynucleotide molecule as described herein. Host cells of the present invention can be any cell capable of producing at least one protein defined herein, and include bacterial, fungal (including yeast), parasite, nematode, arthropod, animal and plant cells. Examples of host cells include Salmonella, Escherichia, Bacillus, Listeria, Saccharomyces, Spodoptera, Mycobacteria, Trichoplusia, BHK (baby hamster kidney) cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusia ni; and non-tumorigenic mouse myoblast G8 cells (e.g., ATCC CRL 1246). Useful yeast cells include Pichia sp., Aspergillus sp. and Saccharomyces sp. Particularly preferred host cells are bacterial cells, fungal cells or plant cells.

In one embodiment, the cell is suitable for fermentation. Examples of useful bacterial cell for fermentation include, but are not limited to, Escherichia sp. (such as Escherichia coli), Bacillus sp. (such as Bacillus subtilis and Bacillus licheniformis), Lactobacillus sp. (such as Lactobacillus brevis), Pseudomonas sp. (such as Pseudomonas aeruginosa) and Streptomyces sp. (Streptomyces lividans). Examples of useful fungal cells for fermentation include, but are not limited to, Candida sp. (such as Candida albicans), Hansenula sp. (such as Hansenula polymorpha), Pichia sp. (Pichia pastoris), Kluveromyces sp. (such as Kluyveromyces marxianus), and Saccharomyces sp. (Saccharomyces cerevisiae).

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules of the present invention include, but are not limited to, operatively linking polynucleotide molecules to high-copy number plasmids, integration of the polynucleotide molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts.

The term “plant” as used herein as a noun refers to a whole plants such as, for example, a plant growing in a field for commercial plant or grain production. A “plant part” refers to vegetative structures (for example, leaves, stems), roots, floral organs/structures, seed (including embryo, endosperm, and seed coat), plant tissue (for example, vascular tissue, ground tissue, and the like), cells and progeny of the same.

A “transgenic plant” refers to a plant that contains a gene construct (“transgene”) not found in a wild-type plant of the same species, variety or cultivar. A “transgene” as referred to herein has the normal meaning in the art of biotechnology and includes a genetic sequence which has been produced or altered by recombinant DNA or RNA technology and which has been introduced into the plant cell. The transgene may include genetic sequences derived from a plant cell. Typically, the transgene has been introduced into the plant by human manipulation such as, for example, by transformation but any method can be used as one of skill in the art recognizes.

A polynucleotide of the present invention may be expressed constitutively in the transgenic plants during all stages of development. Depending on the use of the plant or plant organs, the polypeptides may be expressed in a stage-specific manner. Furthermore, the polynucleotides may be expressed tissue-specifically.

Compositions of the present invention include excipients, also referred to herein as “acceptable carriers”. An excipient can be any material that the animal, plant, plant or animal material, or environment (including soil and water samples) to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal or o-cresol, formalin and benzyl alcohol. Excipients can also be used to increase the half-life of a composition, for example, but are not limited to, polymeric controlled release vehicles, biodegradable implants, liposomes, bacteria, viruses, other cells, oils, esters, and glycols.

Each document, reference, patent application or patent cited in this text is expressly incorporated herein in their entirety by reference, which means that it should be read and considered by the reader as part of this text. That the document, reference, patent application, or patent cited in this text is not repeated in this text is merely for reasons for conciseness. Inclusion does not constitute an admission is made that any of the references constitute prior art or are part of the common general knowledge of those working in the field to which this invention relates.

Optional embodiments of the present invention may also be said to broadly consist in the parts, elements and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth.

It is to be appreciated that reference to “one example” or “an example” of the invention is not made in an exclusive sense. Accordingly, one example may exemplify certain aspects of the invention, whilst other aspects are exemplified in a different example. These examples are intended to assist the skilled person in performing the invention and are not intended to limit the overall scope of the invention in any way unless the context clearly indicates otherwise.

It is to be understood that the terminology employed above is for the purpose of description and should not be regarded as limiting. The described embodiment is intended to be illustrative of the invention, without limiting the scope thereof. The invention is capable of being practised with various modifications and additions as will readily occur to those skilled in the art.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Various substantially and specifically practical and useful exemplary embodiments of the claimed person matter are described herein, textually and/or graphically, including the best mode, if any, known to the inventors for carrying out the claimed person matter.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively and any and all combinations or any two or more of the steps or features.

The inventor(s) expects skilled artisans to employ such variations as appropriate, and the inventor(s) intends for the claimed person matter to be practiced other than as specifically described herein. Accordingly, as permitted by law, the claimed person matter includes and covers all equivalents of the claimed person matter and all improvements to the claimed person matter. Moreover, every combination of the above described elements, activities, and all possible variations thereof are encompassed by the claimed person matter unless otherwise clearly indicated herein, clearly and specifically disclaimed, or otherwise clearly contradicted by context.

The present invention is not to be limited in scope by the specific embodiments described herein, which are intended for the purpose of exemplification only. Functionally equivalent products, compositions and methods are clearly within the scope of the invention as described herein.

The use of any and all examples, or exemplary language (e.g., “such as” or “for example”) provided herein, is intended merely to better illuminate one or more embodiments and does not pose a limitation on the scope of any claimed person matter unless otherwise stated. No language in the specification should be construed as indicating any non-claimed person matter as essential to the practice of the claimed person matter.

Throughout the specification and claims, unless the context requires otherwise, the word “comprise” or variations such as “comprises” or “comprising”, will be understood to imply the inclusion of a stated integer or group of integers but not the exclusion of any other integer or group of integers.

Throughout the specification unless the context requires otherwise, the word “include” or variations such as “includes” or “including”, will be understood to imply the inclusion of a stated integer or group of integers but not the exclusion of any other integer or group of integers.

Moreover, when any number or range is described herein, unless clearly stated otherwise, that number or range is approximate. Recitation of ranges of values herein are merely intended to serve as a shorthand method of referring individually to each separate value falling within the range, unless otherwise indicated herein, and each separate value and each separate sub-range defined by such separate values is incorporated into the specification as if it were individually recited herein. For example, if a range of 1 to 10 is described, that range includes all values there between, such as for example, 1.1, 2.5, 3.335, 5, 6.179, 8.9999, etc., and includes all sub-ranges there between, such as for example, 1 to 3.65, 2.8 to 8.14, 1.93 to 9, etc.

Accordingly, every portion (e.g., title, field, background, summary, description, abstract, drawing figure, etc.) of this application, other than the claims themselves, is to be regarded as illustrative in nature, and not as restrictive; and the scope of person matter protected by any patent that issues based on this application is defined only by the claims of that patent.

While there are shown and described presently further embodiments of the application, it is to be distinctly understood that the application is not limited thereto but may be otherwise variously embodied and practiced within the scope of the following claims.

Any of the features of an embodiment of aspects is applicable to all other aspects and embodiments identified herein. Any of the features of an embodiment is independently combinable, partly or wholly with other embodiments described herein in any way, e.g., one, two, or three or more embodiments may be combinable in whole or in part.

1. A polypeptide comprising:
i) an amino acid sequence as provided in SEQ ID NO:4,
ii) an amino acid sequence which is at least 90% identical to i), or
iii) a biologically active fragment of ii), and
wherein the polypeptide comprises one or both of:
iv) a glutamine (Q) at a position corresponding to amino acid number 221 of SEQ ID NO: 4; and
v) an aspartic acid (D) at a position corresponding to amino acid number 377 of SEQ ID NO:4.

2. The polypeptide according to claim 1, comprising a fusion protein comprising at least one other polypeptide sequence.

3. An isolated and/or exogenous polynucleotide comprising a sequence selected from:
i) a sequence of nucleotides as provided in SEQ ID NO: 2;
ii) a sequence of nucleotides encoding a polypeptide comprising:
iv) an amino acid sequence as provided in SEQ ID NO:4,
v) an amino acid sequence which is at least 90% identical to i), or
vi) a biologically active fragment of ii), and
wherein the polypeptide comprises one or both of:
vii) a glutamine (Q) at a position corresponding to amino acid number 221 of SEQ ID NO:4; and
viii) an aspartic acid (D) at a position corresponding to amino acid number 377 of SEQ ID NO:4,
wherein the polynucleotide is capable of expressing the polypeptide; or iii) a sequence of nucleotides complementary to either i) or ii).

4. The isolated and/or exogenous polynucleotide according to claim 3, wherein the polynucleotide is operably linked to a promoter capable of directing expression of the polypeptide in a cell.

5. The isolated and/or exogenous polynucleotide according to claim 3, wherein the polynucleotide is operably linked to a promoter capable of directing expression of the polypeptide in an expression host cell.

6. A vector comprising the polynucleotide according to claim 3.

7. A nucleic acid construct or expression vector comprising the polynucleotide according to claim 3.

8. A nucleic acid construct or expression vector comprising the polynucleotide of claim 3, wherein the polynucleotide is operably linked to one or more control sequences that direct the production of the polypeptide in an expression host cell.

9. A recombinant expression host cell comprising a polynucleotide encoding the polypeptide according to claim 1, wherein the polynucleotide is operably linked to one or more control sequences that direct the production of the polypeptide.

10. A host cell comprising the polynucleotide according to claim 3.

11. The host cell according to claim 10 comprising a bacterial cell, a fungal cell, or a plant cell.

12. A transgenic non-human organism comprising at least one cell according to claim 10.

13. An extract of the host cell according to claim 10,
wherein the extract comprises the polypeptide.

14. A composition comprising the polypeptide according to claim 1, and one or more acceptable carriers.

15. A composition comprising the extract according to claim 13, and one or more acceptable carriers.

16. (canceled)

17. A method of producing a polypeptide, the polypeptide comprising:
i) an amino acid sequence as provided in SEQ ID NO:4,
ii) an amino acid sequence which is at least 90% identical to i), or
iii) a biologically active fragment of ii), and
wherein the polypeptide comprises one or both of:
iv) a glutamine (Q) at a position corresponding to amino acid number 221 of SEQ ID NO: 4; and
v) an aspartic acid (D) at a position corresponding to amino acid number 377 of SEQ ID NO:4;
wherein the method comprises cultivating a recombinant expression host cell, the recombinant expression host cell comprising an isolated and/or exogenous polynucleotide comprising a sequence selected from:
vi) a sequence of nucleotides as provided in SEQ ID NO: 2;
vii) a sequence of nucleotides encoding the polypeptide; or
viii) a sequence of nucleotides complementary to either i) or ii),
wherein the polynucleotide is operably linked to one or more control sequences that direct the production of the polypeptide under conditions conducive for production of the polypeptide.

18. The method according to claim 17, further comprising recovering the polypeptide.

19. An enzyme comprising the polypeptide according to claim 1.

20. A thermostable enzyme comprising the polypeptide according to claim 1.

21. A hyperthermophilic enzyme comprising the polypeptide according to claim 1.

22. An The enzyme according to claim 19, wherein the enzyme maintains enzymatic activity at a temperature higher than the temperature at which an enzyme comprising a wild-type Thermosyntropha lipolytica Tl_Est47 polypeptide comprising an amino acid sequence as provided in SEQ ID NO:3 loses substantially all enzymatic activity.

23. The enzyme according to claim 19, wherein the enzyme maintains enzymatic activity at 90° C. or higher.

24. The enzyme according to claim 19, wherein the enzyme maintains some enzymatic activity at 95° C. or higher.

25. The enzyme according to claim 19, comprising esterase activity.

26. The enzyme according to claim 19, comprising lipase activity.

27. The polypeptide according to claim 1, comprising a polypeptide variant of wild-type Thermosyntropha lipolytica Tl_Est47, wherein the polypeptide variant of wild-type Thermosyntropha lipolytica Tl_Est47 comprises an amino acid sequence as provided in SEQ ID NO:3.

28. The polynucleotide according to claim 3, being capable of expressing a polypeptide variant of wild-type Thermosyntropha lipolytica Tl_Est47, wherein the wild-type Thermosyntropha lipolytica Tl_Est47 comprises an amino acid sequence expressed by an isolated and/or exogenous polynucleotide comprising a sequence selected from a sequence of nucleotides as provided in SEQ ID NO: 1.

29-31. (canceled)

32. The polynucleotide according to claim 3, wherein the polypeptide comprises a fusion protein comprising at least one other polypeptide sequence.
